SEQUENCE LISTING (51 sequences)

SEQ ID NO: 1 - protein, 213 aa, homo sapiens
MQLPLALCLVCLLVHTAFRVVEGQGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 2 - protein, 462 aa, ARTIFICIAL
  ANTIBODY HEAVY CHAIN
MAWVWTLPFLMAAAQSVQAQVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 237 aa, ARTIFICIAL
  ANTIBODY LIGHT CHAIN
MSVLTQVLALLLLWLTGTRCDIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 4 - protein, 117 aa, ARTIFICIAL
  ANTIBODY VH
QVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSS

SEQ ID NO: 5 - protein, 113 aa, ARTIFICIAL
  ANTIBODY VL
DIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQ

SEQ ID NO: 6 - protein, 10 aa, ARTIFICIAL
  HCDR1
GFTFRSHWLS

SEQ ID NO: 7 - protein, 20 aa, ARTIFICIAL
  HCDR2
WVSNINYDGSSTYYADSVKG

SEQ ID NO: 8 - protein, 8 aa, ARTIFICIAL
  HCDR3
DTYLHFDY

SEQ ID NO: 9 - protein, 14 aa, ARTIFICIAL
  LCDR1
TGTSSDVGDINDVS

SEQ ID NO: 10 - protein, 11 aa, ARTIFICIAL
  LCDR2
LMIYDVNNRPS

SEQ ID NO: 11 - protein, 10 aa, ARTIFICIAL
  LCDR3
QSYAGSYLSE

SEQ ID NO: 12 - protein, 449 aa, ARTIFICIAL
  ANTIBODY HEAVY CHAIN
EVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 214 aa, ARTIFICIAL
  ANTIBODY LIGHT CHAIN
DIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 123 aa, ARTIFICIAL [sequence omitted]

SEQ ID NO: 15 - protein, 107 aa, ARTIFICIAL [sequence omitted]

SEQ ID NO: 16 - protein, 5 aa, ARTIFICIAL
  HCDR1
DYNMH

SEQ ID NO: 17 - protein, 17 aa, ARTIFICIAL
  HCDR2
EINPNSGGAGYNQKFKG

SEQ ID NO: 18 - protein, 14 aa, ARTIFICIAL
  HCDR3
LGYDDIYDDWYFDV

SEQ ID NO: 19 - protein, 11 aa, ARTIFICIAL
  LCDR1
RASQDISNYLN

SEQ ID NO: 20 - protein, 7 aa, ARTIFICIAL
  LCDR2
YTSRLLS

SEQ ID NO: 21 - protein, 9 aa, ARTIFICIAL
  LCDR3
QQGDTLPYT

SEQ ID NO: 22 - protein, 446 aa, ARTIFICIAL
  ANTIBODY HEAVY CHAIN
EVQLVQSGAEVKKPGSSVKVSCKASGFTFTDYIMHWVRQAPGQGLEWMGYINPYNDDTEYNEKFKGRVTITADKSTSTAYMELSSLRSEDTAVYYCARSIYYYDAPFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 213 aa, ARTIFICIAL
  ANTIBODY LIGHT CHAIN
DIQMTQSPSSLSASVGDRVTITCRASQDISSYLNWYQQKPGKAPKLLIYSTSRLNSGVPSRFSGSGSGTDFTLTISSLQPEDFATYYCQQDIKHPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 120 aa, ARTIFICIAL
  ANTIBODY VH
EVQLVQSGAEVKKPGSSVKVSCKASGFTFTDYIMHWVRQAPGQGLEWMGYINPYNDDTEYNEKFKGRVTITADKSTSTAYMELSSLRSEDTAVYYCARSIYYYDAPFAYWGQGTLVTVSS

SEQ ID NO: 25 - protein, 106 aa, ARTIFICIAL
  ANTIBODY VL
DIQMTQSPSSLSASVGDRVTITCRASQDISSYLNWYQQKPGKAPKLLIYSTSRLNSGVPSRFSGSGSGTDFTLTISSLQPEDFATYYCQQDIKHPTFGQGTKVEIK

SEQ ID NO: 26 - protein, 5 aa, ARTIFICIAL
  HCDR1
DYIMH

SEQ ID NO: 27 - protein, 17 aa, ARTIFICIAL
  HCDR2
YINPYNDDTEYNEKFKG

SEQ ID NO: 28 - protein, 11 aa, ARTIFICIAL
  HCDR3
SIYYYDAPFAY

SEQ ID NO: 29 - protein, 11 aa, ARTIFICIAL
  LCDR1
RASQDISSYLN

SEQ ID NO: 30 - protein, 7 aa, ARTIFICIAL
  LCDR2
STSRLNS

SEQ ID NO: 31 - protein, 8 aa, ARTIFICIAL
  LCDR3
QQDIKHPT

SEQ ID NO: 32 - protein, 444 aa, ARTIFICIAL
  ANTIBODY HEAVY CHAIN
QVQLVQSGAEVKKPGSSVKVSCKASGYTFTDYFLNWVRQAPGQGLEWMGTIYPYHDGTTYSQKFKGRVTITADKSTSTAYMELSSLRSEDTAVYYCAREEEDGQFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 33 - protein, 214 aa, ARTIFICIAL
  ANTIBODY LIGHT CHAIN
DIQMTQSPSSLSASVGDRVTITCSASQGIQWYLNWYQQKPGKAPKLLIYYTSSLHSGVPSRFSGSGSGTDFTLTISSLQPEDFATYYCQQHSKLPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 118 aa, ARTIFICIAL
  ANTIBODY VH
QVQLVQSGAEVKKPGSSVKVSCKASGYTFTDYFLNWVRQAPGQGLEWMGTIYPYHDGTTYSQKFKGRVTITADKSTSTAYMELSSLRSEDTAVYYCAREEEDGQFDYWGQGTTVTVSS

SEQ ID NO: 35 - protein, 107 aa, ARTIFICIAL
  ANTIBODY VL
DIQMTQSPSSLSASVGDRVTITCSASQGIQWYLNWYQQKPGKAPKLLIYYTSSLHSGVPSRFSGSGSGTDFTLTISSLQPEDFATYYCQQHSKLPRTFGGGTKVEIK

SEQ ID NO: 36 - protein, 10 aa, ARTIFICIAL
  HCDR1
GYTFTDYFLN

SEQ ID NO: 37 - protein, 17 aa, ARTIFICIAL
  HCDR2
TIYPYHDGTTYSQKFKG

SEQ ID NO: 38 - protein, 9 aa, ARTIFICIAL
  HCDR3
EEEDGQFDY

SEQ ID NO: 39 - protein, 11 aa, ARTIFICIAL
  LCDR1
SASQGIQWYLN

SEQ ID NO: 40 - protein, 7 aa, ARTIFICIAL
  LCDR2
YTSSLHS

SEQ ID NO: 41 - protein, 9 aa, ARTIFICIAL
  LCDR3
QQHSKLPRT

SEQ ID NO: 42 - protein, 444 aa, ARTIFICIAL
  ANTIBODY HEAVY CHAIN
QVQLVQSGAEVKKPGASVKVSCKVSGFPIKDTFQHWVRQAPGKGLEWMGWSDPEIGDTEYASKFQGRVTMTEDTSTDTAYMELSSLRSEDTAVYYCATGDTTYKFDFWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 43 - protein, 214 aa, ARTIFICIAL
  ANTIBODY LIGHT CHAIN
DIQMTQSPSSLSASVGDRVTITCKASQDVHTAVAWYQQKPGKAPKLLIYWASTRWTGVPSRFSGSGSGTDFTLTISSLQPEDFATYYCQQYSDYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 118 aa, ARTIFICIAL [sequence omitted]

SEQ ID NO: 45 - protein, 107 aa, ARTIFICIAL [sequence omitted]

SEQ ID NO: 46 - protein, 10 aa, ARTIFICIAL
  HCDR1
GFPIKDTFQH

SEQ ID NO: 47 - protein, 17 aa, ARTIFICIAL
  HCDR2
WSDPEIGDTEYASKFQG

SEQ ID NO: 48 - protein, 9 aa, ARTIFICIAL
  HCDR3
GDTTYKFDF

SEQ ID NO: 49 - protein, 11 aa, ARTIFICIAL
  LCDR1
KASQDVHTAVA

SEQ ID NO: 50 - protein, 7 aa, ARTIFICIAL
  LCDR2
WASTRWT

SEQ ID NO: 51 - protein, 9 aa, ARTIFICIAL
  LCDR3
QQYSDYPWT

(19) United States
(12) Patent Application Publication
(10) Pub. No.: US 2014/0056912 A1
(43) Pub. Date: Feb. 27, 2014

(54) METHODS OF TREATING SQUAMOUS CELL CARCINOMA

(71) Applicants: Seth Alexander Ettenberg, Melrose, MA (US); Vivien W. Chan, Emeryville, CA (US); Michael Patrick Morrissey, Watertown, MA (US); David Jenkins, Carlisle, MA (US)

(72) Inventors: Seth Alexander Ettenberg, Melrose, MA (US); Vivien W. Chan, Emeryville, CA (US); Michael Patrick Morrissey, Watertown, MA (US); David Jenkins, Carlisle, MA (US)

(73) Assignee: Novartis AG, Basel (CH)

(21) Appl. No.: 14/114,418

(22) PCT Filed: Apr. 27, 2012
(86) PCT No.: PCT/US12/35351

§ 371 Date: Oct. 28, 2013

Related U.S. Application Data
(60) Provisional application No. 61/480,679, filed on Apr. 29, 2011.

(51) Int. Cl. A61K 39/395

(52) U.S. Cl. CPC A61K 39/3955
USPC 424/158.1

(57) ABSTRACT

The disclosure relates to methods for treating a sclerostin-expressing cancer, e.g., a squamous cell carcinoma (SCC), e.g., SCC of the upper aerodigestive tract, esophagus, or lung, employing a therapeutically effective amount of at least one sclerostin antagonist, e.g., an anti-sclerostin antibody, such as Antibody 1, 2, 3, 4 or 5.

### RELATED APPLICATIONS

The application claims priority to U.S. Provisional Patent Application No. 61/480679, filed Apr. 29, 2011, which is incorporated by reference herein in its entirety.

### TECHNICAL FIELD

The disclosure relates to method for treating squamous cell carcinomas (SCC), e.g., SCC of the lung, by antagonizing Sclerostin expression, secretion, signaling and/or function.

### BACKGROUND OF THE DISCLOSURE

Lung cancer is the leading cause of cancer death in women and men worldwide, with metastases to distant organs being the major cause of death for the majority of lung cancer patients. Inamura and Ishikawa (2010) Clin. Exp. Metastasis 27:389-97. Lung cancer is classified as either small-cell lung carcinoma (SCLC) or non-SCLC (NSCLC) types. NSCLC is sub-divided into adenocarcinomas, squamous cell carcinomas (SCC) and large cell carcinomas (LCC). SCC accounts for about 30% of all lung cancers and is the lung cancer most strongly associated with smoking. Platinum-based doublet chemotherapy remains first-line therapy for treating advanced NSCLC patients. G. Selvaggi (2009) Oncology 23:13. However, most data examining the interaction between histology and NSCLC treatment outcome indicates that recently-approved treatment options are less favorable in patients with SCC compared to adenocarcinoma, e.g., the labeling of bevacizumab and pemetrexed is restricted to patients with non-squamous cell NSCLC. Langer (2010) J. Clin. Oncology 28:5311-5320. There are also significant safety risks associated with using bevacizumab or sorafenib to treat NSCLC of a squamous morphology. Langer et al. (2010), supra.

Because SCC is a malignant tumor of the squamous epithelium, it occurs in organs and tissues other than the lung, i.e., organs having squamous epithelium, e.g., head or neck, lung, skin, lips, mouth, esophagus, urinary bladder, prostate, vagina and cervix. SCC is the second most common type of skin cancer and 90% of head or neck cancers are of the SCC type. There are various classifications of SCC, e.g., adenoid squamous-cell carcinoma (Pseudoglandular squamous-cell carcinoma), clear-cell squamous-cell carcinoma (e.g, Clear-cell carcinoma of the skin), spindle-cell squamous-cell carcinoma, signet-ring-cell squamous-cell carcinoma, basaloid squamous-cell carcinoma (e.g., as occurs in the thymus, anus, vulva, vagina, cervix, and aerodigestive tract, e.g., larynx, pharynx, tonsils, tongue, sinus, conjunctiva, nose, ear, submandibular region, esophagus, etc.), verrucous carcinoma, keratoacanthoma. Depending on the type of SCC (effected organ, aggressive or non-aggressive, etc.), the therapy includes surgical removal, chemosurgery, topical medication, radiotherapy, and electrodessication and curettage. The morbidity and discomfort of these treatments for non-lung SCC can be severe.

Given the less favorable treatment and safety outcomes for patients having SCC of the lung (in comparison to other NSCLCs), there is a need for new chemotherapeutic agents to address SCC of the lung. In addition, due to the prevalence of non-lung SCC, e.g, in skin, esophagus, and head and neck cancers, and the discomfort resultant from currently-available therapies, there is a need to design new therapeutic agents that may be used to treat non-lung SCC.

### SUMMARY OF THE DISCLOSURE

A bioinformatics analysis was performed evaluating SOST mRNA expression/gene amplification in normal tissues versus a range of tumors. This analysis spanned not only human patient samples, but also cancer cell lines and tumor models. No significant chromosomal amplifications/deletions were observed, but striking over-expression in a subset of tumors of the squamous subtype, particularly in carcinomas of the lung, esophagus and upper aerodigestive tract was found. Our functional studies included testing anti-sclerostin antibodies in three SCC cell models—two primary tumor models and one cell line. With these studies we show that Antibody 1, when introduced intraveneously into a high-sclerostin expressing primary tumor model of SCC, inhibited tumor growth by 60%.

These findings are surprising, given that sclerostin is a bone anabolic protein, with no recognized role in lung development, maintenance or morphology. Sclerostin was originally identified as a secreted protein that binds BMPs (bone morphogenic proteins), acting as a BMP antagonist in vitro (Winkler et al. (2003) EMBO J. 22(23):6267-76). It is now understood that sclerostin, which is encoded by the SOST gene, acts as a negative regulator of canonical Wnt signaling, either directly by binding to LRP5/LRP6 or indirectly via another mechanism (see, e.g., Mason and Williams (2010) J. Osteoporos Published online Jul. 1, 2010 ID No. 460120; Winkler et al. (2005) J. Biol. Chem. 28;280(4):2498-502). As a result of this function, Sclerostin is a potent negative regulator of bone formation in humans and mice; lack of Sclerostin results in high bone formation, and gives rise to Sclerosteosis and van Buchem disease (Balemans et al. (2001) Hum Mol Genet. 10(5):537-43; Brunkow et al. (2001) Am. J. Hum. Genet. 68(3):577-89; Loots et al. (2005) Genome Res. 15(7):928-35). While aberrant Wnt signaling is implicated in the development of several cancers, prior to this application filing, there was no evidence that Sclerostin is involved in the pathology of SCC (e.g., Tennis et al. (2007) J. Thoracic Oncology 2:889-892; Geryk-Hall and Hughes (2009) Curr. Oncol. Rep. 11:446-53; Niemann et al. (2007) Cancer Res. 67:2916-21). A recent publication, Hu et al. (October 2011) PLoS One 10:e25807, confirms the results reported herein. Hu et al. shows that Wnt pathway components are differentially expressed in SCC samples, i.e., inhibition of the canonical branch of the Wnt pathway, coupled with an enhancement of the noncanonical Wnt PCP signaling cascade. In particular, Hu et al. report that SOST overexpression is enriched in SCC samples, thus confirming our findings.

Accordingly, disclosed herein are methods of treating a squamous cell carcinoma (SCC), comprising administering to a patient (e.g., a human) having SCC a therapeutically effective amount of a sclerostin antagonist (e.g., an anti-sclerostin antibody or antigen-binding fragment thereof, such as Antibody 1, 2, 3, 4 or 5). Also disclosed herein are antagonists of sclerostin for use in treating a squamous cell carcinoma (SCC) (e.g., SCC of the lung, urinary tract, upper aerodigestive tract) in a patient in need thereof Also disclosed herein are uses of antagonists of sclerostin for the manufacture of a medicament for treating a squamous cell carcinoma (SCC) in a patient in need thereof

Additionally disclosed herein are methods of predicting the likelihood that a patient having a SCC will respond to treatment with a sclerostin antagonist, comprising: a) obtaining a biological test sample from said patient; and b) assaying the biological test sample for the magnitude of sclerostin expression, wherein an increase in the magnitude of sclerostin expression in the biological test sample from the patient relative to the magnitude of sclerostin expression in a biological control sample provides an increased likelihood that the patient will respond to treatment of the SCC with the sclerostin antagonist.

Additionally disclosed herein are methods of predicting the likelihood that a patient having a SCC will respond to treatment with a sclerostin antagonist, comprising: assaying a biological test sample from the patient for the magnitude of sclerostin expression, wherein an increase in the magnitude of sclerostin expression in the biological test sample from the patient relative to the magnitude of sclerostin expression in a biological control sample provides an increased likelihood that the patient will respond to treatment of the SCC with the sclerostin antagonist.

Additionally disclosed herein are methods of predicting the likelihood that a patient will develop a SCC, comprising: a) obtaining a biological test sample from said patient; and b) assaying the biological test sample from the patient for the magnitude of sclerostin expression, wherein an increase in the magnitude of sclerostin expression in the biological test sample relative to the magnitude of sclerostin expression in a biological control sample provides an increased likelihood that the patient will develop the SCC.

Additionally disclosed herein are methods of predicting the likelihood that a patient will develop a SCC, comprising assaying a biological test sample from the patient for the magnitude of sclerostin expression, wherein an increase in the magnitude of sclerostin expression in the biological test sample relative to the magnitude of sclerostin expression in a biological control sample provides an increased likelihood that the patient will develop the SCC.

Additionally disclosed herein are methods of treating a SCC in a patient, comprising: a) assaying a biological test sample from the patient for the magnitude of sclerostin expression; and b) selectively administering a sclerostin antagonist to the patent if the magnitude of sclerostin expression in the biological test sample is greater than the magnitude of sclerostin expression in a biological control sample. In some embodiments, these methods further comprise assaying a biological control sample from the patient for the magnitude of sclerostin expression prior to the step of administering.

Additionally disclosed herein are sclerostin antagonists for use in treating a SCC, characterized in that: a) a biological test sample is obtained from a patient having a SCC; b) the biological test sample is assayed for the magnitude of sclerostin expression; and c) a therapeutically effective amount of the sclerostin antagonist is selectively administered to the patient if the biological test sample from the patient has a higher magnitude of sclerostin expression relative to a magnitude of sclerostin expression in a biological control sample.

Additionally disclosed herein are sclerostin antagonists for use in treating a SCC, characterized in that: a) a biological test sample from a patient having a SCC is assayed for the magnitude of sclerostin expression; and b) a therapeutically effective amount of the sclerostin antagonist is selectively administered to the patient if the biological test sample from the patient has a higher magnitude of sclerostin expression relative to a magnitude of sclerostin expression in a biological control sample.

Additionally disclosed herein are kits for use in treating a patient having a SCC, comprising: a) a therapeutically effective amount of a sclerostin antagonist; b) optionally, means for administering said sclerostin antagonist to the patient; c) optionally, at least one additional agent selected from the group consisting of platinum, taxane, EGFR-i, cetuximab, 5-FU, anthracycline, and vinflunine; and d) instructions for administering the sclerostin antagonist to the patient.

Additionally disclosed herein are kits for use in treating a patient having a SCC, comprising: a) a therapeutically effective amount of a sclerostin antagonist; b) at least one probe capable of detecting magnitude of sclerostin expression in a biological test sample from the patient; c) optionally, means for administering the sclerostin antagonist to the patient; and d) instructions for selectively administering the sclerostin antagonist to the patient if the biological test sample from the patient has a higher magnitude of sclerostin expression relative to a magnitude of sclerostin expression in a control sample.

Additionally disclosed herein are kits for use in predicting the likelihood that a patient having a SCC will respond to treatment with a sclerostin antagonist, comprising: a) at least one probe capable of detecting the presence of sclerostin; and b) instructions for using the probe to assay a biological test sample from the patient for the magnitude of sclerostin expression, wherein an increase in the magnitude of sclerostin expression in the biological test sample from the patient relative to a magnitude of sclerostin expression in a biological control sample provides an increased likelihood that the patient will respond to treatment of the SCC with the sclerostin antagonist.

Additionally disclosed herein are kits for use in predicting the likelihood that a patient will develop a SCC, comprising: a) at least one probe capable of detecting the presence of sclerostin; and b) instructions for using the probe to assay a biological test sample from the patient for the magnitude of sclerostin expression, wherein an increase in the magnitude of sclerostin expression in the biological test sample relative to the magnitude of sclerostin expression in a biological control sample provides an increased likelihood that the patient will develop the SCC.

Further embodiments of the disclosed methods, uses, kits and compositions may be found in the Detailed Description of the Disclosure and the Claims.

### BRIEF DESCRIPTION OF THE FIGURES

FIG. 1 is a graph showing SOST mRNA expression in a panel of tumor cell line and primary xenograft samples as determined by quantitative PCR analysis.

FIG. 2 is a graph showing SOST protein expression in a panel of tumor cell line and primary xenograft samples as determined by Western blotting with an anti-SOST antibody. FIG. 3A-B are graphs showing the activity of Antibody 1 in a primary human lung tumor xenograft model, HLUX1726.

FIG. 4 is a graph showing the effect of Antibody 1 on pLRP6 expression in the HLUX1726 xenograft model.

### DETAILED DESCRIPTION OF THE DISCLOSURE

The disclosure provides methods of affecting tumor growth (e.g., slowing, reducing the volume of, reversing, ameliorting, etc.) by inhibiting sclerostin expression, signalling, secretion and/or function in tumors and cancers mediated by inappropriate and/or excessive sclerostin activity (such cancers are referred to herein as “sclerostin-expressing cancers” or “sclerostin-expressing tumors”).

Accordingly, herein are provided, inter alia, methods of treating sclerostin-expressing cancers, e.g., SCC (e.g., SCC of the lung). in a patient (e.g., a human) in need thereof, comprising administering to said patient a therapeutically effective amount of at least one sclerostin antagonist (e.g., an anti-sclerostin antibody or antigen-binding fragment thereof, e.g., Antibody 1, 2, 3, 4 or 5). Also disclosed herein are antagonists of sclerostin for use in treating sclerostin-expressing cancers, e.g., SCC (e.g., SCC of the lung), in a patient in need thereof Further disclosed herein are uses of antagonists of sclerostin for the manufacture of a medicament for treating sclerostin-expressing cancers, e.g., SCC (e.g., SCC of the lung), in a patient in need thereof In preferred embodiments, the sclerostin-expressing cancer is SCC.

Additionally disclosed herein are methods of treating a SCC in a patient, comprising: a) assaying a biological test sample from the patient for the magnitude of sclerostin expression; and b) administering a sclerostin antagonist to the patent if the magnitude of sclerostin expression in the biological test sample is greater than the magnitude of sclerostin expression in a biological control sample. In some embodiments, these methods further comprise assaying a biological control sample from the patient for the magnitude of sclerostin expression prior to the step of administering.

Additionally disclosed herein are sclerostin antagonists for use in treating a SCC, characterized in that: a) a biological test sample is obtained from a patient having a SCC; b) the biological test sample is assayed for the magnitude of sclerostin expression; and c) a therapeutically effective amount of the sclerostin antagonist is administered to the patient if the biological test sample from the patient has a greater magnitude of sclerostin expression relative to a magnitude of sclerostin expression in a biological control sample.

Additionally disclosed herein are sclerostin antagonists for use in treating a SCC, characterized in that: a) a biological test sample from a patient having a SCC is assayed for the magnitude of sclerostin expression; and b) a therapeutically effective amount of the sclerostin antagonist is administered to the SCC patient if the biological test sample from the patient has a greater magnitude of sclerostin expression relative to a magnitude of sclerostin expression in a biological control sample.

In some embodiments of the disclosed methods, uses and kits, the magnitude of sclerostin expression is determined by use of at least one probe capable of detecting the presence of sclerostin. In some embodiments of the disclosed methods, uses and kits, the at least one probe detects a sclerostin nucleic acid or a sclerostin polypeptide. In some embodiments of the disclosed methods, uses and kits, the at least one probe is an anti-sclerostin antibody. As used herein, “squamous cell carcinoma” and “SCC” includes both SCC of the lung and non-lung SCC. SCC can occur in any organ having squamous epithelium, e.g., lung, biliary tract, bone, cervix, endometrium, eye, genital tract, large intestine, oesophagus, ovary, salivary gland, skin, stomach, thymus, upper aerodigestive tract, urinary tract, bladder, prostate, penis, cervix, vagina, vulva, etc. SCC of the lung is cancer of the squamous epithelium that occurs in the lung. Non-lung SCC is cancer of the squamous epithelium that occurs in an organ or tissue other than the lung. In some embodiments of the disclosed methods, kits and uses, the patient has SCC of the lung. In some embodiments of the disclosed methods, kits and uses, the patient has non-lung SCC.

In some embodiments of the disclosed methods, uses or kits, the SCC occurs in the lung, skin, lip, upper aerodigestive tract, urinary tract, esophagus, bladder, prostate, penis, vagina or cervix of the patient. In some embodiments of the disclosed methods, uses or kits, the SCC occurs in the upper aerodigestive tract, esophagus, urinary tract or lung. In some embodiments of the disclosed methods, uses or kits, the SCC occurs in the head or neck, larynx, mouth, pharynx, sinonasal cavity or nasal cavity. In some embodiments of the disclosed methods, uses or kits, the SCC occurs in the orbit, glottis, subglottis, supraglottis, alveolus, alveolus ridge, buccal mucosa, cheek, oral commissure, gingiva, lip, mandible, maxilla, maxillary antrum, mouth floor, mouth roof, paraoral area, retromolar space, oral sulcus, tongue, tonsillar fossa, uvula, oral vestibule, epiglottis, hypopharynx, nasopharnyx, orpharynx or sinus.

As used herein, SCC occurring in the “upper aerodigestive tract” refers to SCC found in the upper respiratory or digestive tract. It includes, inter alia, SCC occurring in the head or neck, larynx, mouth, pharynx, the sinonasal cavity and the nasal cavity. SCC of the upper aerodigestive tract includes SCC occurring in the orbit, glottis, subglottis, supraglottis, alveolus, alveolus ridge, buccal mucosa, cheek, oral commissure, gingiva, lip, mandible, maxilla, maxillary antrum, mouth floor, mouth roof, paraoral area, retromolar space, oral sulcus, tongue, tonsillar fossa, uvula, oral vestibule, epiglottis, hypopharynx, nasopharnyx, orpharynx and sinus.

As used herein, “head or neck” refers to a group of biologically similar cancers within the upper aerodigestive tract, including the lip, oral cavity, nasal cavity, paranasal sinuses, pharynx, and larynx. Cancers of the head and of the neck include tumors of the nasal cavities, paranasal sinuses, oral cavity (e.g., inner lip, tongue floor of mouth, gingivae, hard palate), nasopharynx, oropharynx (e.g., soft palate, base of tongue, tonsils), hypopharynx (pyriform sinuses, posterior pharyngeal wall, postcricoid area) and larynx (e.g., glottic, supraglottic and subglottic cancers). Head and neck cancers have substantial overlap with cancers of the upper aerodigestive tract. For convenience, clinicians commonly refer to these types of cancers collectively as “head and neck” cancers. To alleviate confusion, the instant disclosure uses the phrase “head or neck” rather than “head and neck”. Thus, a patient having head or neck cancer may have, e.g., cancer of the orbit or cancer of the larynx, but need not have both cancer of the orbit and cancer of the larynx. In some embodiments of the disclosed methods and uses, the patient has SCC of the head or neck. As used herein, “sclerostin” is intended to refer to human sclerostin, the amino acid sequence of which is set forth in SEQ ID NO:1.

A patient having a SCC (e.g., SCC of the upper aerodigestive tract, esophagus, urinary tract or lung), suspected of having a SCC, or suspected of developing a SCC in the future would be considered in need of treatment with the disclosed sclerostin antagonists. The terms “treatment”, “treating” or “treat” refer to both prophylactic or preventative treatment as well as curative or disease modifying treatment, including treatment of patient at risk of contracting the cancer or suspected to have contracted the cancer as well as patients who are ill or have been diagnosed as suffering from a cancer or medical condition, and includes suppression of clinical relapse. The treatment may be administered to a subject having a SCC or who ultimately may acquire a SCC, in order to prevent, cure, delay the onset of, reduce the severity of, or ameliorate one or more symptoms of a SCC or recurring SCC, or in order to prolong the survival of a subject beyond that expected in the absence of such treatment. The treatment may be administered to a subject having a SCC to reduce tumor volume or retard tumor growth.

As used herein, the phrase “respond to treatment” refers to an improvement in signs and symptoms of a given disorder following administration of a particular therapy, e.g., a reduction in tumor volume, a retardation of tumor growth, etc. following administration of a sclerostin antagonist (e.g., sclerostin inhibitory polynucleotide, sclerostin inhibitory polypeptide, antagonistic anti-sclerostin antibody or antigen-binding fragments thereof, and antagonistic small molecules).

As used herein, a “therapeutically effective amount” refers to an amount of a sclerostin antagonist (e.g., sclerostin inhibitory polynucleotide, sclerostin inhibitory polypeptide, antagonistic anti-sclerostin antibody or antigen-binding fragments thereof, and antagonistic small molecules, e.g., Antibody 1 as set forth in as disclosed in WO09047356, the contents of which are incorporated by reference herein in its entirety) that is effective, upon single or multiple dose administration to a subject (such as a human patient) at treating, preventing, preventing the onset of, curing, delaying, reducing the severity of, ameliorating at least one symptom of a disorder (e.g., a SCC) or recurring disorder, or prolonging the survival of the subject beyond that expected in the absence of such treatment. When applied to an individual active ingredient (e.g., an anti-sclerostin antibody) administered alone, the term refers to that ingredient alone. When applied to a combination, the term refers to combined amounts of the active ingredients that result in the therapeutic effect, whether administered in combination, serially or simultaneously.

As used herein, the term “patient” includes any human or nonhuman animal. The term “nonhuman animal” includes all vertebrates, e.g., mammals and non-mammals, such as nonhuman primates, sheep, dogs, cats, horses, cows chickens, amphibians, reptiles, etc. In some embodiments of the disclosure, the patient is a human.

The term “biological sample” as used herein refers to a sample from a patient, which may be used for the purpose of identification, diagnosis, prediction, or monitoring. Preferred test and control samples for use in the disclosed methods, uses or kits include tissue derived from the lung, skin, lip, upper aerodigestive tract, urinary tract, esophagus, bladder, prostate, penis, vagina or cervix of a patient. One of skill in the art would realize that some test samples would be more readily analyzed following a fractionation or purification procedure, for example, isolation of DNA from whole blood. Thus, “biological sample” includes the use of, e.g., DNA, RNA and proteins extracted from a patient sample.

As used herein, the phrase “biological test sample” refers to a biological sample obtained from a patient of interest.

In some embodiments of the disclosed methods, uses and kits, the biological test sample is obtained from the lung, skin, lip, upper aerodigestive tract, urinary tract, esophagus, bladder, prostate, penis, vagina or cervix of the patient. In some embodiments of the disclosed methods, uses and kits, the biological test sample is obtained from the head or neck, larynx, mouth, pharynx, sinonasal cavity or nasal cavity. In some embodiments of the disclosed methods, uses and kits, the biological test sample is obtained from the orbit, glottis, subglottis, supraglottis, alveolus, alveolus ridge, buccal mucosa, cheek, oral commissure, gingiva, lip, mandible, maxilla, maxillary antrum, mouth floor, mouth roof, paraoral area, retromolar space, oral sulcus, tongue, tonsillar fossa, uvula, oral vestibule, epiglottis, hypopharynx, nasopharnyx, orpharynx or sinus.

As used herein, the phrase “biological control sample” refers to a biological sample obtained from a standard source. In some embodiments of the disclosed methods, uses and kits, the biological control sample is obtained from a control patient known not to develop SCC. In some embodiments of the disclosed methods, uses and kits, the biological control sample is derived from a tissue within the test patient that does not have SCC. In some embodiments, the control sample may be a reference standard, e.g., the mean level of sclerostin or SOST expression in patients known not to develop SCC.

As used herein, the phrase “patient known not to develop SCC” refers to a patient (e.g., a human patient) who has been previously determined to not suffer from SCC. In some embodiements of the disclosed methods, uses and compositions, the patient is a one known not to develop SCC. In some embodiements of the disclosed methods, uses and compositions, the sample (e.g., biological control sample) is derived from a patient known not to develop SCC.

As used herein, the phrase “derived from a tissue of the patient that does not have SCC” refers to a biological sample taken from a patient who has been determined to not suffer from SCC. Samples from these sources may be used as biological control samples in the disclosed methods. In some embodiements of the disclosed methods, uses and compositions, the sample (e.g., biological control sample) is derived from a tissue of the patient that does not have SCC

In some embodiments of the disclosed methods, uses and kits, the biological control sample is obtained from the lung, skin, lip, upper aerodigestive tract, urinary tract, esophagus, bladder, prostate, penis, vagina or cervix of the patient. In some embodiments of the disclosed methods, uses and kits, the biological control sample is obtained from the head or neck, larynx, mouth, pharynx, sinonasal cavity or nasal cavity. In some embodiments of the disclosed methods, uses and kits, the biological control sample is obtained from the orbit, glottis, subglottis, supraglottis, alveolus, alveolus ridge, buccal mucosa, cheek, oral commissure, gingiva, lip, mandible, maxilla, maxillary antrum, mouth floor, mouth roof, paraoral area, retromolar space, oral sulcus, tongue, tonsillar fossa, uvula, oral vestibule, epiglottis, hypopharynx, nasopharnyx, orpharynx or sinus.

The term “assaying” is used to mean testing and/or measuring. The phrase “assaying the biological sample” and the like is used to mean that a sample may be tested for either the existence or nonexistence of a given substance. It will be understood that, in a situation where the presence of a substance denotes one probability and the absence of a substance denotes a different probabiltity, then either the presence or the absence of such substance may be used to guide a therapeutic decision.

As used herein, the phrase “magnitude of sclerostin expression” refers to the level of a product of the SOST gene in a sample (e.g., the level of expression of a sclerostin nucleic acid or the level of expression of a sclerostin polypeptide). As used herein, the phrase “increase in the magnitude of sclerostin expression” refers to a meaningful increase in the level of expression of sclerostin, e.g., a statistically significant increase.

As used herein, “product of the SOST gene” includes sclerostin nucleic acid products, e.g., mRNA, micro RNAs, fragments of RNAs, etc. and sclerostin polypeptide products, e.g., polypeptides encoded by SOST genes, fragments of polypeptides encoded by SOST genes, etc. As used herein, “a sclerostin polypeptide” refers to a polypeptide encoded by a SOST gene (e.g., a human SOST gene). As used herein, “a sclerostin nucleic acid” refers to any RNA products of the SOST gene and fragments thereof, as well as cDNAs produced from any RNA products of the SOST gene and fragments thereof. In some embodiements of the disclosed methods, uses and compositions, the magnitude of sclerostin expression in a sample is determined by assaying the sample for a product of the SOST gene. In further embodiements of the disclosed methods, uses and compositions, the magnitude of sclerostin expression in a sample is determined by assaying the sample for a sclerostin nucleic acid. In further embodiements of the disclosed methods, uses and compositions, the magnitude of sclerostin expression in a sample is determined by assaying the sample for a sclerostin polypeptide.

As used herein, the term “greater” refers to an amount that is larger in a meaningful way, e.g., a statistically significant difference.

As used herein, “predicting” indicates that the methods described herein provide information to enable a health care provider to determine the likelihood that an individual having SCC will respond to or will respond more favorably to treatment with a sclerostin antagonist, or the likelihood that a patient will eventually develop SCC. It does not refer to the ability to predict response with 100% accuracy. Instead, the skilled artisan will understand that it refers to an increased probability.

As used herein, “likelihood” and “likely” is a measurement of how probable an event is to occur. It may be used interchangably with “probability”. Likelihood refers to a probability that is more than speculation, but less than certainty. Thus, an event is likely if a reasonable person using common sense, training or experience concludes that, given the circumstances, an event is probable. In some embodiments, once likelihood has been ascertained, the patient may be treated (or treatment continued, or treatment proceed with a dosage increase) with the sclerostin antagonist or the patient may not be treated (or treatment discontinued, or treatment proceed with a lowered dose) with the sclerostin antagonist. As used herein, the phrase “likelihood that a patient will develop a SCC” refers to the probability that a patient will become afflicted with SCC.

The phrase “increased likelihood” refers to an increase in the probability that an event will occur. For example, the methods herein allow prediction of whether a patient will display an increased likelihood of responding to treatment with a sclerostin antagonist or an increased likelihood of responding better to treatment with a sclerostin antagonist.

The phrase “decreased likelihood” refers to a decrease in the probability that an event will occur. For example, the methods herein allow prediction of whether a patient will display a decreased likelihood of responding to treatment with a sclerostin antagonist or a decreased likelihood of responding better to treatment with a sclerostin antagonist.

The term “probe” refers to any substance useful for specifically detecting another substance, e.g., a substance related to sclerostin. A probe can be an oligonucleotide or conjugated oligonucleotide that specifically hybridizes to a sclerostin nucleic acid (e.g., mRNA, cDNA). A “conjugated oligonucleotide” refers to an oligonucleotide covalently bound to chromophore or molecules containing a ligand (e.g., an antigen), which is highly specific to a receptor molecule (e.g., an antibody specific to the antigen). The probe can also be a PCR primer, together with another primer, for amplifying a particular region of a sclerostin nucleic acid. Further, the probe can be an antibody that specifically recognizes a sclerostin polypeptide (i.e., by binding an antigen or epitope of sclerostin). In some embodiments of the disclosed methods, uses and compositions, the probe detects a sclerostin nucleic acid. In some embodiements of the disclosed methods, uses and compositions, the probe detects a sclerostin polypeptide. In further embodiements of the disclosed methods, uses and compositions, the probe is an anti-sclerostin antibody, e.g, an antibody as set forth in Table 1, e.g., Antibody 1, 2, 3, 4, or 5.

The term “capable” is used to mean that ability to achieve a given result, e.g., a probe that is capable of detecting the presence of a particular substance means that the probe is able to detect the particular substance.

As used herein, the phrase “capable of detecting the presence of sclerostin” refers to the ability of a probe to provide information regarding the presence (or absence) of a sclerostin nucleic acid or a sclerostin polypeptide in a given sample.

As used herein, the terms “obtain”, “obtained” and “obtaining” means to procure, e.g., to acquire possession of in any way.

As used herein, “selecting” and “selected” in reference to a patient is used to mean that a particular patient is specifically chosen from a larger group of patients on the basis of (due to) the particular patient having a predetermined criteria, e.g., the patient has the presence of, or a particular level of, SOST or sclerostin (e.g., if the magnitude of sclerostin expression in the biological test sample is greater than the magnitude of sclerostin expression in a biological control sample). Similarly, “selectively treating a patient having SCC” refers to providing treatment to a SCC patient that is specifically chosen from a larger group of patients on the basis of (due to) the particular patient having a predetermined criteria, e.g., the patient has the presence of, or a particular level of, SOST or sclerostin (e.g., if the magnitude of sclerostin expression in the biological test sample is greater than the magnitude of sclerostin expression in a biological control sample). Similarly, “selectively administering” refers to administering a drug to a SCC patient that is specifically chosen from a larger group of patients on the basis of (due to) the particular patient having predetermined criteria, e.g., a particular genetic or other biological marker. By selecting, selectively treating and selectively administering, it is meant that a patient is delivered a personalized therapy for SCC based on the patient's biology, rather than being delivered a standard treatment regimen based solely on having SCC.

Sclerostin Antagonists

The instant methods, kits and uses employ sclerostin antagonists (e.g., at least one sclerostin antagonist, e.g., a sclerostin antagonist) for the treatment of a SCC (e.g., SCC of the head or neck, urinary tract, esophagus, or lung). Sclerostin antagonists include, e.g., mouse and human sclerostin inhibitory polynucleotides (i.e., polynucleotides that decrease Sclerostin levels and/or activity either directly or indirectly, e.g., antisense molecules, siRNAs, aptamers); sclerostin inhibitory polypeptides (i.e., polypeptides that decrease sclerostin levels and/or activity either directly or indirectly, e.g., fragments of sclerostin, such as soluble fragments containing the BMP and/or LRP-interaction domains, and fusion proteins thereof); antagonistic anti-sclerostin antibodies or antigen-binding fragments thereof (i.e., antibodies or antigen-binding antibody fragments that decrease sclerostin activity and/or expression either directly or indirectly, including antagonistic antibodies and antigen-binding fragments thereof that bind full-length sclerostin and/or sclerostin fragments); and antagonistic small molecules (e.g., siRNAs, aptamers, and small inorganic and/or organic molecules or compounds), which may be used to suppress sclerostin activity, signaling, expression and/or secretion, and which may be used in diagnosing, prognosing, monitoring, treating, ameliorating and/or preventing a SCC (e.g., SCC of the upper aerodigestive tract, urinary tract, esophagus, or lung) related to increased sclerostin activity, expression and/or secretion.

In preferred embodiments, the sclerostin antagonists for use in the disclosed uses and methods are anti-sclerostin antibodies or antigen-binding fragments thereof An antibody is a polypeptide comprising a framework region from an immunoglobulin gene or portion thereof that specifically binds and recognizes an epitope, e.g., an epitope found on sclerostin. The term “antibody” as used herein includes whole antibodies and any antigen-binding fragment or single chains thereof A whole “antibody” is a glycoprotein comprising at least two heavy (H) chains and two light (L) chains inter-connected by disulfide bonds. Each heavy chain is comprised of a heavy chain variable (V_{H}) region and a heavy chain constant region. The heavy chain constant region is comprised of three domains, CH_{L}, CH2 and CH3. Each light chain is comprised of a light chain variable (V_{L}) region and a light chain constant region. The light chain constant region is comprised of one domain, CL. The V_{L} and V_{H} regions can be further subdivided into regions of hypervariability, termed complementarity determining regions (CDR), interspersed with regions that are more conserved, termed framework regions (FR). Each V_{L} and V_{H} is composed of three CDRs and four FRs arranged from amino-terminus to carboxy-terminus in the following order: FR1, CDR1, FR2, CDR2, FR3, CDR3, FR4. The CDRs of the heavy chain are referred to herein as HCDR1, HCDR2 and HCDR3. The CDRs of the light chain are referred to herein as LCDR1, LCDR2, and LCDR3. The variable regions of the heavy and light chains contain a binding domain that interacts with an epitope on an antigen. The constant regions of the antibodies may mediate the binding of the immunoglobulin to host tissues or factors, including various cells of the immune system (e.g., effector cells) and the first component (Clq) of the classical complement system.

The term “antibody” includes single domain antibodies, maxibodies, nanobodies, peptibodies (Amgen), minibodies, intrabodies, diabodies, triabodies, tetrabodies, v-NAR and bis-scFv (see, e.g., Hollinger & Hudson, Nature Biotechnology, 23, 9, 1126-1136 (2005)). Antigen-binding fragments of antibodies can be grafted into scaffolds based on polypeptides such as Fibronectin type III (Fn3) (see U.S. Pat. No. 6,703,199, which describes fibronectin polypeptide monobodies). Details of various types of antibodies and antigen-binding fragments thereof for use in the disclosed methods may be found in WO09047356.

Also included within the definition of “antibody” are single-chain antibodies. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding region” of an antibody. A single-chain antibody may comprise the antibody variable regions alone, or in combination, with all or part of the following polypeptide elements: hinge region, CH1, CH2, and CH3 domains of an antibody molecule.

Also included within the definition of “antibody” are antigen-binding fragments of antibodies. It has been shown that the antigen-binding function of an antibody can be performed by fragments of a full-length antibody. Thus, “antigen-binding fragment” refers to one or more fragments of an antibody that retain the ability to specifically bind to an antigen (e.g., an antigen of sclerostin). Antigen-binding fragments include, e.g., but are not limited to, Fab, Fab′ and F(ab′)₂, Fd, single-chain Fvs (scFv), single-chain antibodies, disulphide-linked Fvs (sdFv) and fragments comprising either a V_{L} or V_{H} domain. Examples include: (i) a Fab fragment, a monovalent fragment consisting of the V_{L}, V_{H}, CL and CH1 domains; (ii) a F(ab′) 2 fragment, a bivalent fragment comprising two Fab fragments linked by a disulphide bridge at the hinge region; (iii) a Fd fragment consisting of the V_{H} and CH1 domains; (iv) a Fv fragment consisting of the V_{L} and VH domains of a single arm of an antibody, (v) a dAb fragment (Ward et al., Nature 341: 544-546, 1989; Muyldermans et al., TIBS 24: 230-235, 2001), which consists of a V_{H} domain; and (vi) an isolated complementarity determining region (CDR). Also included within the definition of “antibody” are any combinations of variable regions and hinge region, CH1, CH2, and CH3 domains. Antigen binding fragments can be incorporated into single chain molecules comprising a pair of tandem Fv segments (VH-CH1-VH-CH1) which, together with complementary light chain polypeptides, form a pair of antigen binding regions (Zapata et al., Protein Eng. 8(10):1057-1062 (1995); and U.S. Pat. No. 5,641,870). Antibody fragments are obtained using conventional techniques known to those of skill in the art, and the fragments are screened for antigen-binding capability in the same manner as are whole antibodies.

It will be understood by one skilled in the art that antibodies may undergo a variety of posttranslational modifications. The type and extent of these modifications often depends on the host cell line used to express the protein as well as the culture conditions. Such modifications may include variations in glycosylation, methionine oxidation, diketopiperizine formation, aspartate isomerization and asparagine deamidation. A frequent modification is the loss of a carboxy-terminal basic residue (such as lysine or arginine) due to the action of carboxypeptidases (as described in Harris, RJ. Journal of Chromatography 705:129-134, 1995). Table 1, infra, provides antibodies for use in the disclosed used, methods and kits that may retain or relinquish the carboxy-terminal lysine.

Antibody constant regions may be of various isotypes. “Isotype” refers to the antibody class (e.g., IgM, IgE, IgG such as IgG₁, IgG₄ or IgG₂) that is provided by the heavy chain constant region genes. In some embodiments of the disclosed methods and uses, the sclerostin antagonist is an anti-sclerostin antibody of the IgG₁, IgG₄ or IgG₂ isotype.

The terms “monoclonal antibody” as used herein refer to an antibody molecule derived from a preparation of antibody molecules of single molecular composition. Thus, a monoclonal antibody displays a single binding specificity and affinity for a particular epitope. In some embodiments of the disclosed methods and uses, the sclerostin antagonist is a monoclonal anti-sclerostin antibody.

Chimeric or humanized antibodies of the present disclosure can be prepared using art-recognized techniques employing the sequences of the antibodies and antibody fragments described herein (e.g., see Table 1). DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al. In some embodiments of the disclosed methods and uses, the sclerostin antagonist is a chimeric or humanized anti-sclerostin antibody

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis as described in Knappik, et al. (2000. J Mol Biol 296, 57-86). The human antibodies for use in the disclosed methods may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences, which are instead referred to as “chimeric” antibodies and/or “humanized” humanized antibodies. In some embodiments of the disclosed methods, pharmaceutical compositions, kits and uses, the sclerostin antagonist is a human antibody.

The term “human monoclonal antibody” refers to antibodies displaying a single binding specificity and that have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell. Such animals are available from the companies Medarex and Kim. In another embodiment, the human monoclonal antibodies are produced by a transgenic mouse having human immunoglobulin genes. In some embodiments of the disclosed methods, pharmaceutical compositions, kits and uses, the sclerostin antagonist is a human monoclonal antibody.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo. In some embodiments of the disclosed methods, pharmaceutical compositions, kits and uses, the sclerostin antagonist is a recombinant human antibody.

As used herein, an antibody that “specifically binds to a sclerostin polypeptide” is intended to refer to an antibody that binds to sclerostin polypeptide with a K_{D} of about 1×10⁻⁸ M or less, about 1×10⁻⁹ M or less, or about 1×10⁻¹⁰ M or less. An antibody that “cross-reacts with an antigen other than sclerostin” (or the like) is intended to refer to an antibody that binds to that antigen with a K_{D} of about 0.5×10⁻⁸ M or less, about 5×10⁻⁹ M or less, or about 2×10⁻⁹ M or less. An antibody that “does not cross-react with a particular antigen” (or the like) is intended to refer to an antibody that binds to a particular antigen with a K_{D} of about 1.5×10⁻⁸ M or greater, or a K_{D} of between about 5×10⁻⁸ M and about 10 x 10⁻⁸ M, or about 1×10⁻⁷ M or greater. Antibodies that do not cross-react with a particular antigen exhibit a lack of significant binding against that particular antigen in standard binding assays. In certain embodiments of the disclosed methods and uses, the anti-sclerostin antibody specifically binds sclerostin. In certain embodiments of the disclosed methods, pharmaceutical compositions, kits and uses, the anti-sclerostin antibody specifically binds sclerostin and does not cross react with an antigen other than sclerostin. In certain embodiments of the disclosed methods, pharmaceutical compositions, kits and uses, the anti-sclerostin antibody specifically binds sclerostin and does not cross react with Dan or Gremlin.

In some embodiments of the disclosed methods, compositions, uses and kits, the anti-sclerostin antibody competes with Antibody 1, 2, 3, 4 or 5 for binding to sclerostin. Competing antibodies typically recognize the same epitope. Thus, e.g., in some embodiments of the disclosed methods, compositions, uses and kits, the anti-sclerostin antibody binds the same epitope as that which is bound by Antibody 1, 2, 3, 4, or 5. The sclerostin epitopes bound by Antibodies 1, 2, 3, 4 and 5 are set forth, e.g., in U.S. Pat. No. 7,758,858, U.S. Pat. No. 7,381,409, U.S. Pat. No. 7,578,999, WO05003158, WO06119062, WO06119107, WO08115732, and U.S. Pat. No. 7,744,874, the contents of which are incorporated by reference herein in their entirety.

The term “about” in relation to a numerical value x means, for example, +/−10%.

As used herein, “significant” in relation to a numerical value refers to statistical significance.

As used herein, “a cell-based Wnt signaling assay” is intended to refer to a cell-based (e.g., HEK293) super top flash (STF) assay. Such assay is described in more details in WO09047356. In certain embodiments of the disclosed methods, pharmaceutical compositions, kits and uses, the antibodies have an IC₅₀ less than about 1 μM, preferably less than about 100 nM and more preferably less than about 20 nM as measured in a cell-based Wnt signaling assay in HEK293 cell lines in the presence of sclerostin.

In one embodiment, anti-sclerostin antibodies for use in the disclosed methods, pharmaceutical compositions, kits and uses have the ability to reverse sclerostin inhibition of in vitro bone mineralization. In a related embodiment, they have the ability to reverse sclerostin inhibition of the Wnt-1 mediated signaling pathway. In another related embodiment, they disrupt sclerostin LRP6 binding and can block the inhibitory effect that sclerostin has at high doses on BMP induced Smadl phosphorylation.

Sclerostin inhibits Wntl -mediated activation of STF (Supertopflash, reporter readout for canonical Wnt signaling) in HEK293 cells. In some embodiments, the antibodies for use in the disclosed methods, pharmaceutical compositions, kits and uses restore the Wnt signaling reporter readout in a highly reproducible manner.

The observed inhibitory effect of the antibodies according to the disclosure on sclerostin action in the Wnt signaling reporter assay in non-osteoblastic cells has been shown to translate into induction of bone formation responses due to sclerostin inhibition in vivo. Indeed, in vivo experiments in aged rodents show that the antibodies according to the disclosure promote strong bone anabolism. The bone mass increase reached the effect level of daily intermittent treatment with extremely high anabolic doses of parathyroid hormone (which was used as a positive control). Therefore, according to another embodiment, the antibodies for use in the disclosed methods, pharmaceutical compositions, kits and uses have affinities to sclerostin in the low pM range (preferably about 100 pM or less, preferably about 50 pM or less, preferably about 10 pM or less, more preferably about 1 pM or less) and inhibit sclerostin impact on wnt signalling with an IC₅₀ around about 10 nM.

As used herein, a “BMP2-induced mineralization assay” is intended to refer to an assay the measures restoration of BMP2 induced mineralisation in the presence of sclerostin in a cell-based assay (e.g., in MC3T3 cells). Such assay is described in more details in WO09047356. In certain embodiments of the disclosed methods, pharmaceutical compositions, kits and uses, the antibodies have an IC₅₀ less than about 1 μM, preferably less than about 500 nM and more preferably less than about 200 nM as measured in BMP2-induced mineralization assay in MC3T3 cells in the presence of sclerostin.

As used herein, a “Smadl phosphorylation assay” is intended to refer to an assay the measures restoration of BMP6 induced Smadl phosphorylation in the presence of sclerostin in a cell based assay (e.g., in MC3T3-E1 cells). Such assay is described in more details in WO09047356. In certain embodiments of the disclosed methods, pharmaceutical compositions, kits and uses, the antibodies have an IC₅₀ less than about 1 μM, preferably less than about 500 nM, preferably less than about 200 nM as measured in BMP6 Smadl phosphorylation assay in MC3T3-E1 cell line in the presence of sclerostin

As used herein, an “LRP6/sclerostin ELISA” is intended to refer to an ELISA assay used to measure the interaction of sclerostin with LRP-6. Such assay is described in more details in WO09047356. In certain embodiments of the disclosed methods, pharmaceutical compositions, kits and uses, the antibodies have an IC₅₀ less than about 1 μM, preferably less than about 100 nM, more preferably less than about 10 nM (e.g., about 6 nM), more preferably less than about SnM, more preferably less than about 3 nM as measured in LRP6/sclerostin ELISA. In certain embodiments of the disclosed methods and uses, the antibodies have an IC₅₀ of about 5.8 nM, about 6.0 nM, about 6.5 nM, about 7.0 nM, about 9.6 nM, about 10.6 nM, about 12.1 nM, or about 19.4 nM in a in LRP6/sclerostin ELISA.

An antibody that “inhibits” one or more sclerostin functional properties (e.g., biochemical, immunochemical, cellular, physiological or other biological activities, or the like, as described above, e.g., BMP-2 induced mineralization) as determined according to methodologies known to the art and described herein, will be understood to relate to a statistically significant decrease in the particular activity relative to that seen in the absence of the antibody (or when a control antibody of irrelevant specificity is present). An antibody that inhibits sclerostin activity effects such a statistically significant decrease by at least 10% of the measured parameter, by at least 50%, 80% or 90%, and in certain embodiments an antibody used in the disclosed methods, pharmaceutical compositions, kits and uses may inhibit greater than 95%, 98% or 99% of sclerostin functional activity.

The term “K_{assoc}” or “Kₐ”, as used herein, is intended to refer to the association rate of a particular antibody-antigen interaction, whereas the term “K_{dis}” or “K_{D},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor system, such as a ^{Biacore}® system, KinExA-based system, Electrochemiluminescene (BioVeris), Solution Equilibrium Titration, Receptor Binding Inhibition Potency Assay, etc. These assays are set forth in detail in WO09047356, WO06119107 and U.S. Pat. No. 7,744,874.

As used herein, “affinity” refers to the strength of interaction between an antibody and an antigen at a single antigenic site. Within each antigenic site, the variable region of the antibody “arm” interacts through weak non-covalent forces with antigen at numerous sites; the more interactions, the stronger the affinity.

As used herein, “avidity” refers to an informative measure of the overall stability or strength of the antibody-antigen complex. It is controlled by three major factors: antibody affinity; the valence of both the antigen and antibody; and the structural arrangement of the interacting parts. Ultimately these factors define the specificity of the antibody, that is, the likelihood that the particular antibody is binding to a precise antigen epitope.

As used herein, “high affinity” for an IgG antibody refers to an antibody having a K_{D} of about 10⁻⁸ M or less, about 10⁻⁹ M or less, or about 10⁻¹⁰ M or less for a target antigen. However, “high affinity” binding can vary for other antibody isotypes. For example, “high affinity” binding for an IgM isotype refers to an antibody having a K_{D} of about 10⁻⁷ M or less, or about 10⁻⁸ M or less. In some embodiments of the disclosed methods, pharmaceutical compositions, kits and uses, the sclerostin antagonist is high affinity anti-sclerostin antibody.

In some embodiments, antibodies for use in the disclosed methods, pharmaceutical compositions, kits and uses have a K_{D} less than about 10 nM, less than about 1 nM, less than about 100 pM, less than about 50 pM, or less than about 25 pM, e.g., about 15-25 pM, e.g., about 21 pM +/−4 pM as determined by surface plasmon resonance. In some embodiments, antibodies for use in the disclosed methods, pharmaceutical compositions, kits and uses have a K_{D} of about 0.5 to about 10 pM, e.g, about 0.6 pM, about 1 pM, about 3 pM, about 4 pM, or about 6 pM as measured in a KinExA-based determination experiment as set forth in Example 10 of WO06119107. In some embodiments, antibodies for use in the disclosed methods, pharmaceutical compositions, kits and uses have a K_{D} of about 0.2 to about 2.5 pM, e.g, about 0.3 pM, about 0.6 pM, about 2.2 pM as measured in a KinExA-based determination experiment as set forth in Example 3 of U.S. Pat. No. 7,744,874.

Various sclerostin antagonists (e.g., anti-sclerostin antibodies) are disclosed in WO09047356, WO2000/32773, WO2006102070, US20080227138, US20100028335, US 20030229041, WO2005003158, WO2009039175 WO2009079471, WO03106657, WO2006119062, WO08115732, WO2005/014650, WO2005/003158, WO2006/119107, WO2008/061013, WO2008/133722, WO2008/115732, U.S. Pat. No. 7,592,429, U.S. Pat. No. 7,879,322, U.S. Pat. No. 7,744,874, the contents of which are incorporated by reference herein in their entirety. Any (or several) of the sclerostin antagonists disclosed in these references may be used in the disclosed methods, pharmaceutical compositions, kits and uses. Further anti-sclerostin antibodies that may be used in the disclosed methods and uses include those known as AMG167 and AMG785 (Amgen) (see, e.g., Padhi et al. (2011) J. Bone Miner. Res. 26:19-26) and those found in Ominsky et al. (2010) J. Bone Min. Res (Epub Dec. 2); Li et al. (2010) J. Bone Miner. Res. 25:2371-80; Li et al. (2009) J. Bone Miner Res. 24:578-88; Ominsky et al. (2010) J. Bone Miner Res. 25:948-59. In some embodiments, an anti-sclerostin antibody for use in the disclosed methods and uses binds to an epitope of sclerostin described in WO2006/119062, WO2005014650 or WO2005003158 or WO09047356.

Preferred anti-sclerostin antibodies and antigen binding fragments thereof for use in the disclosed methods, pharmaceutical compositions, kits and uses are found in WO09047356 (equivalent to U.S. Pat. No. 7,879,322), WO06119107 (equivalent to U.S. Pat. No. 7,872,106 and US 7592429) and WO08115732 (equivalent to U.S. Pat. No. 7,744,874), e.g.:

TABLE 1
Preferred anti-sclerostin antibodies for use in the disclosed methods,
pharmaceutical compositions, kits and uses. The CDR regions in
Table 1 are delineated using the Kabat system (Kabat, E. A., et al.,
1991 Sequences of Proteins of Immunological Interest, Fifth Edition,
U.S. Department of Health and Human Services, NIH
Publication No. 91-3242).

| | | Sequence and |
|---|---|---|
| | Sequence and Designation for | Designation of |
| | this Disclosure | Reference Publications |
| | Antibody 1 | WO09047356 |
| Heavy | SEQ ID NO: 2 (with or without the | SEQ ID NO: 114 |
| chain (H) | 19 amino acid signal peptide) | |
| Light | SEQ ID NO: 3 (with or without 20 | SEQ ID NO: 125 |
| chain (L) | amino acid signal peptide) | |
| V_{H} | SEQ ID NO: 4 | SEQ ID NO: 70 |
| V_{L} | SEQ ID NO: 5 | SEQ ID NO: 81 |
| HCDR1 | SEQ ID NO: 6 | SEQ ID NO: 4 |
| HCDR2 | SEQ ID NO: 7 | SEQ ID NO: 15 |
| HCDR3 | SEQ ID NO: 8 | SEQ ID NO: 26 |
| LCDR1 | SEQ ID NO: 9 | SEQ ID NO: 37 |
| LCDR2 | SEQ ID NO: 10 | SEQ ID NO: 48 |
| LCDR3 | SEQ ID NO: 11 | SEQ ID NO: 59 |
| | | “Ab-5” of |
| | Antibody 2 | WO2006119107 |
| H | SEQ ID NO: 12 (with or without | SEQ ID NO: 145 |
| | carboxy-terminal lysine) | |
| L | SEQ ID NO: 13 | SEQ ID NO: 141 |
| V_{H} | SEQ ID NO: 14 | SEQ ID NO: 378 |
| V_{L} | SEQ ID NO: 15 | SEQ ID NO: 376 |
| HCDR1 | SEQ ID NO: 16 | SEQ ID NO: 245 |
| HCDR2 | SEQ ID NO: 17 | SEQ ID NO: 246 |
| HCDR3 | SEQ ID NO: 18 | SEQ ID NO: 247 |
| LCDR1 | SEQ ID NO: 19 | SEQ ID NO: 78 |
| LCDR2 | SEQ ID NO: 20 | SEQ ID NO: 79 |
| LCDR3 | SEQ ID NO: 21 | SEQ ID NO: 80 |
| | | “Ab-23” of |
| | Antibody 3 | WO2006119107 |
| H | SEQ ID NO: 22 (with or without | SEQ ID NO: 345 |
| | carboxy-terminal lysine) | |
| L | SEQ ID NO: 23 | SEQ ID NO: 341 |
| V_{H} | SEQ ID NO: 24 | SEQ ID NO: 366 |
| V_{L} | SEQ ID NO: 25 | SEQ ID NO: 364 |
| HCDR1 | SEQ ID NO: 26 | SEQ ID NO: 269 |
| HCDR2 | SEQ ID NO: 27 | SEQ ID NO: 270 |
| HCDR3 | SEQ ID NO: 28 | SEQ ID NO: 271 |
| LCDR1 | SEQ ID NO: 29 | SEQ ID NO: 239 |
| LCDR2 | SEQ ID NO: 30 | SEQ ID NO: 240 |
| LCDR3 | SEQ ID NO: 31 | SEQ ID NO: 241 |
| | | “Antibody 86” of |
| | Antibody 4 | U.S. Pat. No. 7,744,874 |
| H | SEQ ID NO: 32 | SEQ ID NO: 2 |
| L | SEQ ID NO: 33 | SEQ ID NO: 5 |
| V_{H} | SEQ ID NO: 34 | SEQ ID NO: 14 |
| V_{L} | SEQ ID NO: 35 | SEQ ID NO: 17 |
| HCDR1 | SEQ ID NO: 36 | SEQ ID NO: 20 |
| HCDR2 | SEQ ID NO: 37 | SEQ ID NO: 21 |
| HCDR3 | SEQ ID NO: 38 | SEQ ID NO: 22 |
| LCDR1 | SEQ ID NO: 39 | SEQ ID NO: 23 |
| LCDR2 | SEQ ID NO: 40 | SEQ ID NO: 24 |
| LCDR3 | SEQ ID NO: 41 | SEQ ID NO: 25 |
| | | “Antibody 88” of |
| | Antibody 5 | U.S. Pat. No. 7,744,874 |
| H | SEQ ID NO: 42 | SEQ ID NO: 3 |
| L | SEQ ID NO: 43 | SEQ ID NO: 6 |
| V_{H} | SEQ ID NO: 44 | SEQ ID NO: 15 |
| V_{L} | SEQ ID NO: 45 | SEQ ID NO: 18 |
| HCDR1 | SEQ ID NO: 46 | SEQ ID NO: 26 |
| HCDR2 | SEQ ID NO: 47 | SEQ ID NO: 27 |
| HCDR3 | SEQ ID NO: 48 | SEQ ID NO: 28 |
| LCDR1 | SEQ ID NO: 49 | SEQ ID NO: 29 |
| LCDR2 | SEQ ID NO: 50 | SEQ ID NO: 30 |
| LCDR3 | SEQ ID NO: 51 | SEQ ID NO: 31 |

Given that the antibodies used in the disclosed methods, pharmaceutical compositions, kits and uses can bind to sclerostin and that antigen-binding specificity is provided primarily by the CDR1, 2 and 3 regions, the VH CDR1, 2 and 3 sequences and VL CDR1, 2 and 3 sequences can be “mixed and matched” (i.e., CDRs from different antibodies can be mixed and matched), although each antibody contains a HCDR1, HCDR2 and HCDR3, as well as a LCDR1, LCDR2 and LCDR3 to create other anti-sclerostin antibodies. Sclerostin binding of such “mixed and matched” antibodies can be tested using the binding assays described in WO2009/047356. When V_{H} CDR sequences are mixed and matched, the HCDR1, HCDR2 and/or HCDR3 sequence from a particular V_{H} sequence should be replaced with a structurally similar CDR sequence(s). Likewise, when V_{L} CDR sequences are mixed and matched, the LCDR1, LCDR2 and/or LCDR3 sequence from a particular V_{L} sequence should be replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences shown herein (e.g., Table 1) for monoclonal antibodies that may be used in the disclosed methods, pharmaceutical compositions, kits and uses.

Particularly preferred antibodies for use with the disclosed methods, pharmaceutical compositions, kits and uses are an anti-sclerostin antibodies disclosed in WO09047356 (the complete contents of which are incorporated herein by reference). In one embodiment, the anti-sclerostin antibody for use in the disclosed methods and uses is found in WO09047356 and referred to herein as “Antibody 1” (See Table 1). Antibody 1 has a V_{H} domain with amino acid SEQ ID NO:4 and a V_{L} domain with amino acid SEQ ID NO:5. Other anti-sclerostin antibodies useful with the present disclosure may include one or more (1, 2, 3, 4, 5 or 6) CDRs from Antibody 1. The CDRs in the heavy chain are SEQ ID NOs: 6-8. The CDRs in the light chain are SEQ ID NOs:9-11. The Antibody 1 V_{H} CDRs may be expressed along with V_{H} framework regions (e.g., V_{H} human framework regions), the Antibody 1 V_{L} CDRs may be expressed along with V_{L} framework regions (e.g., V_{L} human framework regions), the Antibody 1 V_{H} and V_{L} CDRs may be expressed along with V_{H} and V_{L} framework regions (e.g., V_{H} and V_{L} human framework regions) (e.g., human or humanized), and the Antibody 1 variable domains may be expressed as SEQ ID NOs:2 and 3.

Disclosed herein are methods, uses and kits for affecting tumor growth (e.g., slowing, reducing the volume of, reversing, ameliorting, etc.) by inhibiting sclerostin expression and/or function in tumors and cancers mediated by inappropriate and/or excessive sclerostin activity. In preferred embodiments, the cancer mediated by inappropriate and/or excessive sclerostin activity is SCC.

In some of the disclosed, methods, uses and kits, the sclerostin antagonist is an anti-sclerostin antibody or antigen-binding fragment thereof In some embodiments, the anti-sclerostin antibody or antigen-binding fragment thereof: binds to human sclerostin with a K_{D} less than 10 nM as determined by surface plasmon resonance or a biosensor system; has an IC₅₀ less than 1 μM as measured in a cell-based Wnt signaling assay in HEK293 cell lines in the presence of sclerostin; has an IC₅₀ less than 1 μM as measured in BMP2-induced mineralization assay in MC3T3 cells in the presence of sclerostin; has an IC₅₀ less than 1 μM as measured in LRP6/sclerostin ELISA; and/or has an IC₅₀ less than 1 n,M as measured in BMP6 Smadl phosphorylation assay in MC3T3-E1 cell line in the presence of sclerostin.

In some of the disclosed, methods, uses and kits, the anti-sclerostin antibody or antigen-binding fragment thereof is selected from the group consisting of: an anti-sclerostin antibody or antigen-binding fragment thereof comprising a heavy chain variable region comprising the amino acid sequence set forth as SEQ ID NO:4; an anti-sclerostin antibody or antigen-binding fragment thereof comprising a light chain variable region comprising the amino acid sequence set forth as SEQ ID NO:5; an anti-sclerostin antibody or antigen-binding fragment thereof comprising a heavy chain variable region comprising the amino acid sequence set forth as SEQ ID NO:4 and a light chain variable region comprising the amino acid sequence set forth as SEQ ID NO:5; or an anti-sclerostin antibody or antigen-binding fragment thereof comprising the three Complementarity-Determining Regions (CDRs) of the amino acid sequence set forth as SEQ ID NO:4 and the three CDRs of the amino acid sequence set forth as SEQ ID NO:5.

In further embodiments, the three CDRs of the amino acid sequence set forth as SEQ ID NO:4 are set forth in SEQ ID NO:6, SEQ ID NO:7, and SEQ ID NO:8. In further embodiments the three CDRs of the amino acid sequence set forth as SEQ ID NO:5 are set forth in SEQ ID NO:9, SEQ ID NO:10, and SEQ ID NO:11.

In some of the disclosed, methods, uses and kits, the anti-sclerostin antibody or antigen-binding fragment thereof is selected from the group consisting of: an anti-sclerostin antibody or antigen-binding fragment thereof comprising a heavy chain variable region comprising the amino acid sequence set forth as SEQ ID NO:14; an anti-sclerostin antibody or antigen-binding fragment thereof comprising a light chain variable region comprising the amino acid sequence set forth as SEQ ID NO:15; an anti-sclerostin antibody or antigen-binding fragment thereof comprising a heavy chain variable region comprising the amino acid sequence set forth as SEQ ID NO:14 and a light chain variable region comprising the amino acid sequence set forth as SEQ ID NO:15; or an anti-sclerostin antibody or antigen-binding fragment thereof comprising the three CDRs of the amino acid sequence set forth as SEQ ID NO:14 and the three CDRs of the amino acid sequence set forth as SEQ ID NO:15.

In further embodiments, the three CDRs of the amino acid sequence set forth as SEQ ID NO:14 are set forth in SEQ ID NO:16, SEQ ID NO:17, and SEQ ID NO:18. In further embodiments, the three CDRs of the amino acid sequence set forth as SEQ ID NO:15 are set forth in SEQ ID NO:19, SEQ ID NO:20, and SEQ ID NO:21.

In some of the disclosed, methods, uses and kits, the anti-sclerostin antibody or antigen-binding fragment thereof is selected from the group consisting of: an anti-sclerostin antibody or antigen-binding fragment thereof comprising a heavy chain variable region comprising the amino acid sequence set forth as SEQ ID NO:24; an anti-sclerostin antibody or antigen-binding fragment thereof comprising a light chain variable region comprising the amino acid sequence set forth as SEQ ID NO:25; an anti-sclerostin antibody or antigen-binding fragment thereof comprising a heavy chain variable region comprising the amino acid sequence set forth as SEQ ID NO:24 and a light chain variable region comprising the amino acid sequence set forth as SEQ ID NO:25; or an anti-sclerostin antibody or antigen-binding fragment thereof comprising the three CDRs of the amino acid sequence set forth as SEQ ID NO:24 and the three CDRs of the amino acid sequence set forth as SEQ ID NO:25.

In further embodiments, the three CDRs of the amino acid sequence set forth as SEQ ID NO:24 are set forth in SEQ ID NO:26, SEQ ID NO:27, and SEQ ID NO:28. In further embodiments, the three CDRs of the amino acid sequence set forth as SEQ ID NO:25 are set forth in SEQ ID NO:29, SEQ ID NO:30, and SEQ ID NO:31.

In some of the disclosed, methods, uses and kits, the anti-sclerostin antibody or antigen-binding fragment thereof is selected from the group consisting of: an anti-sclerostin antibody or antigen-binding fragment thereof comprising a heavy chain variable region comprising the amino acid sequence set forth as SEQ ID NO:34; an anti-sclerostin antibody or antigen-binding fragment thereof comprising a light chain variable region comprising the amino acid sequence set forth as SEQ ID NO:35; an anti-sclerostin antibody or antigen-binding fragment thereof comprising a heavy chain variable region comprising the amino acid sequence set forth as SEQ ID NO:34 and a light chain variable region comprising the amino acid sequence set forth as SEQ ID NO:35; or an anti-sclerostin antibody or antigen-binding fragment thereof comprising the three CDRs of the amino acid sequence set forth as SEQ ID NO:34 and the three CDRs of the amino acid sequence set forth as SEQ ID NO:35.

In further embodiments, the three CDRs of the amino acid sequence set forth as SEQ ID NO:34 are set forth in SEQ ID NO:36, SEQ ID NO:37, and SEQ ID NO:38. In further embodiments, the three CDRs of the amino acid sequence set forth as SEQ ID NO:35 are set forth in SEQ ID NO:39, SEQ ID NO:40, and SEQ ID NO:41.

In some of the disclosed, methods, uses and kits, the anti-sclerostin antibody or antigen-binding fragment thereof is selected from the group consisting of: an anti-sclerostin antibody or antigen-binding fragment thereof comprising a heavy chain variable region comprising the amino acid sequence set forth as SEQ ID NO:44; an anti-sclerostin antibody or antigen-binding fragment thereof comprising a light chain variable region comprising the amino acid sequence set forth as SEQ ID NO:45; an anti-sclerostin antibody or antigen-binding fragment thereof comprising a heavy chain variable region comprising the amino acid sequence set forth as SEQ ID NO:44 and a light chain variable region comprising the amino acid sequence set forth as SEQ ID NO:45; or an anti-sclerostin antibody or antigen-binding fragment thereof comprising the three CDRs of the amino acid sequence set forth as SEQ ID NO:44 and the three CDRs of the amino acid sequence set forth as SEQ ID NO:45.

In further embodiments, the three CDRs of the amino acid sequence set forth as SEQ ID NO:44 are set forth in SEQ ID NO:46, SEQ ID NO:47, and SEQ ID NO:48. In further embodiments, the three CDRs of the amino acid sequence set forth as SEQ ID NO:45 are set forth in SEQ ID NO:49, SEQ ID NO:50, and SEQ ID NO:51.

In some of the disclosed, methods, uses and kits, the anti-sclerostin antibody or antigen-binding fragment thereof is an anti-sclerostin antibody. In some of the disclosed, methods, uses and kits, the anti-sclerostin antibody is a chimeric antibody, a humanized antibody, or a human antibody. In some of the above methods and uses, the anti-sclerostin antibody is a monoclonal anti-sclerostin antibody or a human recombinant anti-sclerostin antibody. In some of the disclosed, methods, uses and kits, the anti-sclerostin antibody is of the IgG₁, IgG₂ or IgG₄ isotype.

In some of the disclosed, methods, uses and kits, the anti-sclerostin antibody or antigen-binding fragment thereof is an antigen-binding fragment of an antibody. In some of the disclosed, methods, uses and kits, the antigen-binding fragment comprises an F(ab′)₂, Fab, Fab′, Fv, Fc or Fd fragment.

### Pharmaceutical Compositions

In practicing the methods of treatment or uses of the present disclosure, a therapeutically effective amount of at least one sclerostin antagonist (e.g., an anti-sclerostin antibody) is administered to a patient, e.g., a mammal (e.g., a human), having a SCC (e.g., SCC of the upper aerodigestive tract, urinary tract, esophagus, or lung).

A sclerostin antagonist (e.g., an anti-sclerostin antibody, such as Antibody 1, 2, 3, 4 or 5) may be administered in accordance with the methods and uses of the disclosure either alone or in combination with other agents (e.g., one or more additional agents) and therapies, such as, e.g., in combination with additional chemotherapeutic agents and cheomtherapeutic regimens or in combination with additional sclerostin antagonists. When coadministered with one or more agents, the sclerostin antagonist (e.g., an anti-sclerostin antibody) may be administered either simultaneously with the other agent, or sequentially. If administered sequentially, the attending physician will decide on the appropriate sequence of administering the sclerostin antagonist (e.g., an anti-sclerostin antibody) in combination with other agents. Additional agents for use in combination with the disclosed sclerostin antagonists include, e.g., platinum, taxane, EGFR-i (EGFR-mutated LC), cetuximab (EGFR-amplified LC), combined radiation/platinum, combined radiation/cetuximab, 5-FU, anthracycline, vinflunine, REOLYSIN®, carboplatin, paclitaxel, bevacizumab, gefitinib, capecitabine, erlotinib, pemetrexed, sorafenib, metesanib, cediranib, etoposide, dexrazozxane, G-CSF, PEG-filgrastim, mesna, leucovorin, mifamurtide, endostar, everolimus, sorafenib, bisphosphonates (e.g, zoledronic acid), figitumumab, cetuximab, sirolimus, rapamycin, Affinitor®, temsirolimus, interferon alphas (2 a or 2 b, including pegylated and nonpegylated forms), pamidronate, thiotepa, rexin G, L-MTP-PE, glucarpidase, sargramostim, bevacizumab, Nimotuzumab, docetaxel, 5-flourouracil, rosuvastatin, toxotere, azacitidine, sulindak, API31510, ZD1839, ribavirin, celecoxib, Pazopanib (GW786034), ARQ510, panitumumab, romidepsin, gemcytibine, OncoLar, pemetrexed, trabectedin, LY2523355, VM4-037, MLN8237, zileuton, motexafin gadolinium, methotrexate, cisplatin, trastuzumab, doxorubicin, ifosfamide, VEGF-receptor inhibitors (AZD2171), cyclophosphamide, vincristine, topotectan, IGF receptor antagonists (e.g., 19D12 [SCH 717454]), MEK 1/2 inhibitors (e.g., AZD6244), proteosome inhibitors (e.g., bortezomib), BCL-2 inhibitors (e.g., ABT-263), aurora A kinase inhibitors (e.g., MLN8237), histone deacetylase inhibitors (e.g., vorinostat), EGFR and ERBB2 inhibitors (e.g., lapitinib), sunitinib, liposomal muramyl-tripeptide phosphatidyl ethanolamine (L-MTP-PE), ifosfamide, and combinations thereof.

When a therapeutically effective amount of a sclerostin antagonist (e.g., an anti-sclerostin antibody) is administered orally, the binding agent will be in the form of a tablet, capsule, powder, solution or elixir. When administered in tablet form, the pharmaceutical composition of the disclosure may additionally contain a solid carrier such as a gelatin or an adjuvant. When administered in liquid form, a liquid carrier such as water, petroleum, oils of animal or plant origin such as peanut oil (exercising caution in relation to peanut allergies), mineral oil, soybean oil, or sesame oil, or synthetic oils may be added. The liquid form of the pharmaceutical composition may further contain components such as physiological saline solution, dextrose or other saccharide solution, or glycols such as ethylene glycol, propylene glycol, or polyethylene glycol.

When a therapeutically effective amount of a sclerostin antagonist (e.g., an anti-sclerostin antibody) is administered by intravenous, cutaneous or subcutaneous injection, the sclerostin antagonist will be in the form of a pyrogen-free, parenterally acceptable solution. A pharmaceutical composition for intravenous, cutaneous, or subcutaneous injection may contain, in addition to the sclerostin antagonist, an isotonic vehicle such as sodium chloride, Ringer's, dextrose, dextrose and sodium chloride, lactated Ringer's, or other vehicle as known in the art.

Pharmaceutical compositions for use in the disclosed methods may be manufactured in conventional manner. In one embodiment, the pharmaceutical composition is preferably provided in lyophilized form. For immediate administration it is dissolved in a suitable aqueous carrier, for example sterile water for injection or sterile buffered physiological saline. If it is considered desirable to make up a solution of larger volume for administration by infusion rather than a bolus injection, may be advantageous to incorporate human serum albumin or the patient's own heparinized blood into the saline at the time of formulation. The presence of an excess of such physiologically inert protein prevents loss of antibody by adsorption onto the walls of the container and tubing used with the infusion solution. If albumin is used, a suitable concentration is from about 0.5 to about 4.5% by weight of the saline solution. Other formulations comprise liquid or lyophilized formulation.

The appropriate dosage will, of course, vary depending upon, for example, the particular sclerostin antagonist to be employed, the host, the mode of administration and the nature and severity of the condition being treated, and on the nature of prior treatments that the patient has undergone. Ultimately, the attending health care provider will decide the amount of the sclerostin antagonist with which to treat each individual subject. In some embodiments, the attending health care provider may administer low doses of the sclerostin antagonist and observe the subject's response. In other embodiments, the initial dose(s) of sclerostin antagonist administered to a subject are high, and then are titrated downward until signs of relapse occur. Larger doses of the sclerostin antagonist may be administered until the optimal therapeutic effect is obtained for the subject, and at that point the dosage is not generally increased further.

A sclerostin antagonist is conveniently administered parenterally, intravenously, e.g. into the antecubital or other peripheral vein, intramuscularly, or subcutaneously. The duration of intravenous (i.v.) therapy using a pharmaceutical composition of the present disclosure will vary, depending on the severity of the disease being treated and the condition and personal response of each individual patient. Also contemplated is subcutaneous (s.c.) therapy using a pharmaceutical composition of the present disclosure. The health care provider will decide on the appropriate duration of i.v. or s.c. therapy and the timing of administration of the therapy, using the pharmaceutical composition of the present disclosure.

Satisfactory results (treatment, prophylaxis, delay of onset of symptoms, etc.) are generally indicated to be obtained at dosages from about 0.05 mg to about 30 mg per kilogram body weight, more usually from about 0.1 mg to about 20 mg per kilogram body weight. The frequency of dosing may be in the range from about once per day up to about once every three months, e.g., in the range from about once every 2 weeks up to about once every 12 weeks, e.g., once every four to eight weeks. The dosing frequency will depend on, inter alia, the phase of the treatment regimen. In some embodiments, the anti-sclerostin antibody dose may be from about 1 mg/kg to about 500 mg/kg, or about 10 mg/kg to about 400 mg/kg, or about 100 mg/kg to about 350 mg/kg, or about 200 mg/kg to about 300 mg/kg.

Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1 to about 1000 μg/ml and in some methods about 25 to about 300 μg/ml.

Alternatively, antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. Pegylation technology may be used to increase the antibody half-life. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

For some anti-sclerostin antibodies, e.g., Antibody 1, 2, 3, 4 or 5, the dose may be about 5 mg/kg to about 300 mg/kg, or about 10 mg/kg to about 200 mg/kg, or about 20 mg/kg to about 100 mg/kg, or about 30 mg/kg to about 50 mg/kg. In preferred embodiments, the anti-sclerostin antibody, e.g., Antibody 1, may be administered as about 20 mg/kg. In some embodiments, the anti-sclerostin antibody, e.g., Antibody 1, 2, 3, 4 or 5, is administered subcutaneously as about 0.1, about 0.3, about 1, about 3, about 5, or about 10 mg/kg or intravenously as about 1 or about 5 mg/kg. In some embodiments, the anti-sclerostin antibody—e.g., Antibody 1, 2, 3, 4 or 5, is administered daily, twice in a week, weekly, every other week, monthly, every other month, quarterly, every six months, or yearly. In some embodiments, the anti-sclerostin antibody, e.g., Antibody 1, 2, 3, 4 or 5, is administered singly (i.e., only once) or multiply.

“mg/kg” means mg drug per kg body weight of the patient to be treated.

In one embodiment, the total dose of anti-sclerostin antibody given to a patient over the course of a year may be about 500 mg to about 50,000 mg, or about 1000 mg to about 10,000 mg.

In some methods, pharmaceutical compositions, kits and uses, two or more anti-sclerostin antibodies, e.g., with the same or with different binding specificities (e.g., binding the same epitope but having a different binding affinity or binding a different epitope) are administered simultaneously or sequentially (with or without additional agents), in which case the dosage of each antibody administered falls within the ranges indicated.

In some of the disclosed, methods, uses and kits, the sclerostin antagonist is administered with at least one additional agent (e.g., chemotherapeutic agent) selected from the group consisting of platinum, taxane, EGFR-i (EGFR-mutated LC), cetuximab (EGFR-amplified LC), 5-FU, anthracycline, and vinflunine.

### Diagnostic Methods

The present disclosure is based, in part, on the discovery of striking over-expression of sclerostin in a subset of tumors of the squamous subtype, particularly in carcinomas of the lung, esophagus and upper aerodigestive tract. As such, the magnitude of sclerostin expression (e.g., sclerostin protein, sclerostin mRNA levels) may be used as a marker for the development of SCC, as well as a marker of the likelihood that an SCC patient will respond favorably to treatment with a sclerostin antagonist (e.g., an anti-sclerostin antibody or antigen-binding fragment thereof, such as Antibody 1, 2, 3, 4 or 5).

Accordingly, disclosed herein are methods of predicting the likelihood that a patient having a SCC will respond to treatment with a sclerostin antagonist, comprising: a) obtaining a biological test sample from said patient; and b) assaying the biological test sample for the magnitude of sclerostin expression, wherein an increase in the magnitude of sclerostin expression in the biological test sample from the patient relative to the magnitude of sclerostin expression in a biological control sample provides an increased likelihood that the patient will respond to treatment of the SCC with the sclerostin antagonist.

Additionally disclosed herein are methods of predicting the likelihood that a patient having a SCC will respond to treatment with a sclerostin antagonist, comprising: assaying a biological test sample from the patient for the magnitude of sclerostin expression, wherein an increase in the magnitude of sclerostin expression in the biological test sample from the patient relative to the magnitude of sclerostin expression in a biological control sample provides an increased likelihood that the patient will respond to treatment of the SCC with the sclerostin antagonist.

Additionally disclosed herein are methods of predicting the likelihood that a patient will develop a SCC, comprising: a) obtaining a biological test sample from said patient; and b) assaying the biological test sample from the patient for the magnitude of sclerostin expression, wherein an increase in the magnitude of sclerostin expression in the biological test sample relative to the magnitude of sclerostin expression in a biological control sample provides an increased likelihood that the patient will develop the SCC.

Additionally disclosed herein are methods of predicting the likelihood that a patient will develop a SCC, comprising assaying a biological test sample from the patient for the magnitude of sclerostin expression, wherein an increase in the magnitude of sclerostin expression in the biological test sample relative to the magnitude of sclerostin expression in a biological control sample provides an increased likelihood that the patient will develop the SCC.

In some embodiments of the disclosed predictive methods, the magnitude of sclerostin expression is determined by use of at least one probe capable of detecting the presence of sclerostin. In some embodiments of the disclosed predictive methods, the at least one probe detects a sclerostin nucleic acid or a sclerostin polypeptide. In some embodiments of the disclosed predictive methods, the at least one probe is an anti-sclerostin antibody.

In some embodiments of the disclosed predictive methods, the biological control sample is obtained from a control patient known not to develop SCC. In some embodiments of the disclosed predictive methods, the biological control sample is derived from a tissue of the patient that does not have SCC.

In some embodiments of the disclosed predictive methods, the biological control sample is obtained from the lung, skin, lip, upper aerodigestive tract, urinary tract, esophagus, bladder, prostate, penis, vagina or cervix of the patient. In some embodiments of the disclosed predictive methods, the biological control sample is obtained from the head or neck, larynx, mouth, pharynx, sinonasal cavity or nasal cavity. In some embodiments of the disclosed predictive methods, the biological control sample is obtained from the orbit, glottis, subglottis, supraglottis, alveolus, alveolus ridge, buccal mucosa, cheek, oral commissure, gingiva, lip, mandible, maxilla, maxillary antrum, mouth floor, mouth roof, paraoral area, retromolar space, oral sulcus, tongue, tonsillar fossa, uvula, oral vestibule, epiglottis, hypopharynx, nasopharnyx, orpharynx or sinus.

In some embodiments of the disclosed predictive methods, the biological test sample is obtained from the lung, skin, lip, upper aerodigestive tract, urinary tract, esophagus, bladder, prostate, penis, vagina or cervix of the patient. In some embodiments of the disclosed predictive methods, the biological test sample is obtained from the head or neck, larynx, mouth, pharynx, sinonasal cavity or nasal cavity. In some embodiments of the disclosed predictive methods, the biological test sample is obtained from the orbit, glottis, subglottis, supraglottis, alveolus, alveolus ridge, buccal mucosa, cheek, oral commissure, gingiva, lip, mandible, maxilla, maxillary antrum, mouth floor, mouth roof, paraoral area, retromolar space, oral sulcus, tongue, tonsillar fossa, uvula, oral vestibule, epiglottis, hypopharynx, nasopharnyx, orpharynx or sinus.

In some embodiments of the disclosed predictive methods, the SCC occurs in the lung, skin, lip, upper aerodigestive tract, urinary tract, esophagus, bladder, prostate, penis, vagina or cervix of the patient. In some embodiments of the disclosed predictive methods, the SCC occurs in the upper aerodigestive tract, esophagus, urinary tract or lung. In some embodiments of the disclosed predictive methods, the SCC occurs in the head or neck, larynx, mouth, pharynx, sinonasal cavity or nasal cavity. In some embodiments of the predictive methods, the SCC occurs in the orbit, glottis, subglottis, supraglottis, alveolus, alveolus ridge, buccal mucosa, cheek, oral commissure, gingiva, lip, mandible, maxilla, maxillary antrum, mouth floor, mouth roof, paraoral area, retromolar space, oral sulcus, tongue, tonsillar fossa, uvula, oral vestibule, epiglottis, hypopharynx, nasopharnyx, orpharynx or sinus.

### Kits and Articles of Manufacture

Disclosed herein are also kits (i.e., an article of manufacture) useful for treating a SCC (e.g., SCC of the upper aerodigestive tract, urinary tract, esophagus, or lung) and/or predicting the likelihood that a patient having a SCC will respond to treatment with a sclerostin and/or predicting the likelihood that a patient will develop a SCC Such kits may comprise at least one sclerostin antagonist, e.g. an anti-sclerostin antibody, e.g., e.g., Antibody 1, 2, 3, 4 or 5, preferably Antibody 1, or a pharmaceutical composition comprising at least one sclerostin antagonist, e.g. an anti-sclerostin antibody, e.g., Antibody 1. Such kits may comprise means for administering the sclerostin antagonist (e.g., a syringe, an autoinjector or a prefilled pen) and instructions for use. These kits may contain additional therapeutic agents for treating a SCC (e.g., SCC of the upper aerodigestive tract, urinary tract, esophagus, or lung), for delivery in combination with the enclosed sclerostin antagonist(s), e.g., an anti-sclerostin antibody, e.g., Antibody 1, 2, 3, 4 or 5, preferably Antibody 1.

Accordingly, provided herein are kits for use in treating a patient having a SCC, comprising: a) a therapeutically effective amount of a sclerostin antagonist; b) optionally, means for administering said sclerostin antagonist to the patient; c) optionally, at least one additional agent selected from the group consisting of platinum, taxane, EGFR-i, cetuximab, 5-FU, anthracycline, and vinflunine; and d) instructions for administering the sclerostin antagonist to the patient.

Additionally disclosed herein are kits for use in treating a patient having a SCC, comprising: a) a therapeutically effective amount of a sclerostin antagonist; b) at least one probe capable of detecting the magnitude of sclerostin expression in a biological test sample from the patient; c) optionally, means for administering the sclerostin antagonist to the patient; and d) instructions for administering the sclerostin antagonist to the patient if the biological test sample from the patient has a greater magnitude of sclerostin expression relative to a magnitude of sclerostin expression in a control test sample.

Additionally disclosed herein are kits for use in predicting the likelihood that a patient having a SCC will respond to treatment with a sclerostin antagonist, comprising: a) at least one probe capable of detecting the presence of sclerostin; and b) instructions for using the probe to assay a biological test sample from the patient for the magnitude of sclerostin expression, wherein an increase in the magnitude of sclerostin expression in the biological test sample from the patient relative to a magnitude of sclerostin expression in a biological control sample provides an increased likelihood that the patient will respond to treatment of the SCC with the sclerostin antagonist.

Additionally disclosed herein are kits for use in predicting the likelihood that a patient will develop a SCC, comprising: a) at least one probe capable of detecting the presence of sclerostin; and b) instructions for using the probe to assay a biological test sample from the patient for the magnitude of sclerostin expression, wherein an increase in the magnitude of sclerostin expression in the biological test sample relative to the magnitude of sclerostin expression in a biological control sample provides an increased likelihood that the patient will develop the SCC.

In some embodiments of the disclosed kits, the magnitude of sclerostin expression is determined by use of at least one probe capable of detecting the presence of sclerostin. In some embodiments of the disclosed kits, the at least one probe detects a sclerostin nucleic acid or a sclerostin polypeptide. In some embodiments of the disclosed kits, the at least one probe is an anti-sclerostin antibody.

In some embodiments of the disclosed kits, the biological control sample is obtained from a control patient known not to develop SCC. In some embodiments of the disclosed kits, the biological control sample is derived from a tissue of the patient that does not have SCC.

In some embodiments of the disclosed kits, the biological control sample is obtained from the lung, skin, lip, upper aerodigestive tract, urinary tract, esophagus, bladder, prostate, penis, vagina or cervix of the patient. In some embodiments of the disclosed kits, the biological control sample is obtained from the head or neck, larynx, mouth, pharynx, sinonasal cavity or nasal cavity. In some embodiments of the disclosed kits, the biological control sample is obtained from the orbit, glottis, subglottis, supraglottis, alveolus, alveolus ridge, buccal mucosa, cheek, oral commissure, gingiva, lip, mandible, maxilla, maxillary antrum, mouth floor, mouth roof, paraoral area, retromolar space, oral sulcus, tongue, tonsillar fossa, uvula, oral vestibule, epiglottis, hypopharynx, nasopharnyx, orpharynx or sinus.

In some embodiments of the disclosed kits, the biological test sample is obtained from the lung, skin, lip, upper aerodigestive tract, urinary tract, esophagus, bladder, prostate, penis, vagina or cervix of the patient. In some embodiments of the disclosed kits, the biological test sample is obtained from the head or neck, larynx, mouth, pharynx, sinonasal cavity or nasal cavity. In some embodiments of the disclosed kits, the biological test sample is obtained from the orbit, glottis, subglottis, supraglottis, alveolus, alveolus ridge, buccal mucosa, cheek, oral commissure, gingiva, lip, mandible, maxilla, maxillary antrum, mouth floor, mouth roof, paraoral area, retromolar space, oral sulcus, tongue, tonsillar fossa, uvula, oral vestibule, epiglottis, hypopharynx, nasopharnyx, orpharynx or sinus.

In some embodiments of the disclosed kits, the at least one probe detects a sclerostin nucleic acid or a sclerostin polypeptide. In some embodiments of the disclosed kits, the at least one probe is an anti-sclerostin antibody.

In some embodiments of the disclosed kits, the SCC occurs in the lung, skin, lip, upper aerodigestive tract, urinary tract, esophagus, bladder, prostate, penis, vagina or cervix of the patient. In some embodiments of the disclosed methods kits, the SCC occurs in the upper aerodigestive tract, esophagus, urinary tract or lung. In some embodiments of the disclosed kits, the SCC occurs in the head or neck, larynx, mouth, pharynx, sinonasal cavity or nasal cavity. In some embodiments of the disclosed kits, the SCC occurs in the orbit, glottis, subglottis, supraglottis, alveolus, alveolus ridge, buccal mucosa, cheek, oral commissure, gingiva, lip, mandible, maxilla, maxillary antrum, mouth floor, mouth roof, paraoral area, retromolar space, oral sulcus, tongue, tonsillar fossa, uvula, oral vestibule, epiglottis, hypopharynx, nasopharnyx, orpharynx or sinus.

All patents, published patent applications, publications, references and other material referred to in this disclosure are incorporated by reference herein in their entirety.

### EXAMPLES

### Example 1

### Expression Data Generation and Integration

Expression data was generated and analyzed using the OncExpress database, which integrates Affymetrix U133plus2 data from Novartis, NCBI GEO, EBI ArrayExpress, and additional sources. Data for all samples were subjected to the Affymetrix MAS5 algorithm, in a manner consistent across all samples. Samples were normalized to a 2% trimmed mean of 150. Sample annotations were curated to conform to the COSMIC ontology, which includes hierarchical levels of annotation on primary site and histology.

Data obtained from primary human tumor xenografts was generated in the following manner: tumor specimens were collected in RPMI supplemented with 1% penicillin/streptomycin from patients during surgical resection with ischemic time less than one hour. Fragments of 15-30 mm³ free of necrotic tissue were grafted subcutaneously into interscapular fat pad of 6- to 8-week-old female nude mice under isoflurane anesthesia. Mice were maintained in specific pathogen-free animal housing and handled in accordance with the Novartis Animal Care and Use Committee protocols and regulations. Xenografts appeared at the graft site 2 to 8 months after grafting. They were subsequently transplanted from mouse to mouse once tumors reached 700-800 mm³ until a reasonably consistent growth rate is achieved. Frozen stocks in RPMI supplemented with 50% FBS and 10% DMSO were generated during serial passage in mice and were tested to ensure successful establishment of a xenograft model.

Fragments of 30-50 mg from patients and xenografts at each passage were snap frozen for gene expression profiling, copy number as well as mutation analyses. Fragments of 150 mg of each successfully engrafted xenograft model were also collected and subject to histological analysis. An established tumor xenograft model was further used for in vivo studies after passage four. For gene expression profiling, total RNA was isolated using affinity resin (QIAGEN RNeasy Mini Kit; QIAGEN AG). RNA integrity and purity were assessed with the RNA 6000 Nano LabChip system on a Bioanalyzer 2100 (Agilent Technologies).

### Example 2

### Expression Analysis of SOST

Data were extracted from Oncexpress for SOST (probe set 223869_at), and divided into the following categories:

Human Squamous Cell Carcinoma Primary Tumors (Defined by “Histology Subtype 1”=“Squamous cell carcinoma”) at the following “primary sites”:
Lung squamous cell carcinoma
Esophageal squamous cell carcinoma
Upper aerodigestive tract squamous cell carcinoma
Urinary tract squamous cell carcinoma

Represented in the upper aerodigestive tract (primary sites) group were samples from head or neck, larnyx, mouth, pharynx, sinonasal and nasal cavity (Subtype 1), with further subtypes (Subtype 2) found in orbit, glottis, subglottis, supraglottis, alveolus, alveolus ridge, buccal mucosa, cheek, oral commissure, gingiva, lip, mandible, maxilla, maxillary antrum, mouth floor, mouth roof, paraoral area, retromolar space, oral sulcus, tongue, tonsillar fossa, uvula, oral vestibule, epiglottis, hypopharynx, nasopharnyx, orpharynx or sinus.

### Human Non-Squamous Primary Tumors:

All available human non-squamous primary tumors at all primary sites

### Normal Human Tissues:

Lung

Esophagus
Upper aerodigestive tract
Urinary tract
   Primary human tumor xenografts passaged in mice
Lung squamous cell carcinoma
All non-squamous tumors
   No other squamous cell xenografts primary sites were available with more than one sample.
   Expression of SOST across these categories is summarized in Table 2:

TABLE 2
SOST sample expression

| | | | | Mean |
|---|---|---|---|---|
| Histology | Primary Site | Sample Type | N | Expression |
| squamous | lung | primary tumor | 157 | 350.3 |
| squamous | esophagus | primary tumor | 11 | 198.3 |
| squamous | upper aerodigestive | primary tumor | 204 | 52.6 |
| | tract | | | |
| squamous | urinary tract | primary tumor | 4 | 84.7 |
| all (except | all | primary tumor | 15524 | 14.1 |
| squamous) | | | | |
| n/a | lung | normal | 66 | 8.6 |
| n/a | esophagus | normal | 6 | 8.7 |
| n/a | upper aerodigestive | normal | 70 | 11.5 |
| | tract | | | |
| n/a | urinary tract | normal | 9 | 12.4 |
| squamous | lung | primary tumor | 39 | 387.0 |
| | | xenograft | | |
| all (except | all | primary tumor | 234 | 13.6 |
| squamous) | | xenograft | | |

In order to assess the suitability of SOST as a potential target in squamous cell carcinoma (SCC), the expression of SOST in each of categories 1-4 above were calculated with respect to category 5, and also with respect to categories 6-9 of the matched primary site. Statistical significance was assessed using a homoscedastic, 2-tailed t-test on log-transformed expression values; p-values <0.05 are considered significant. Results, shown as fold change, are summarized in Table 3:

TABLE 3
Analysis of suitability of SOST as a potential target in SCC

| | | | Fold |
|---|---|---|---|
| Histology | Analysis | p-value | Change |
| primary tumor | squamous lung vs normal lung | 1.9E−12 | 40.77 |
| primary tumor | squamous lung vs (all except | 3.5E−73 | 24.76 |
| | squamous) | | |
| primary tumor | squamous esophagus vs normal | 1.4E−02 | 22.89 |
| | esophagus | | |
| primary tumor | squamous esophagus vs (all | 3.4E−08 | 14.02 |
| | except squamous) | | |
| primary tumor | squamous UAT* vs normal UAT | 2.1E−05 | 4.58 |
| primary tumor | squamous UAT vs (all except | 2.1E−32 | 3.72 |
| | squamous) | | |
| primary tumor | squamous urinary tract vs | 1.3E−01 | 6.81 |
| | normal urinary tract | | |
| primary tumor | squamous urinary tract vs | 1.7E−02 | 5.99 |
| | (all except squamous) | | |
| primary tumor | squamous lung vs (all except | 2.0E−08 | 28.44 |
| xenograft | squamous) | | |

*UAT = refers to upper aerodigestive tract (pharynx, head and neck, mouth)

### Example 3

### Conclusions from Expression Studies

1) For each of four primary sites of squamous cell tumors (lung, esophagus, upper aerodigestive tract, urinary tract), SOST expression is significantly higher than in non-squamous tumors.

2) For three of four primary sites of squamous cell primary tumors (lung, esophagus, upper aerodigestive tract), SOST expression is significantly higher than in the respective normal tissue. Urinary tract squamous cell carcinoma has 6.8-fold higher SOST expression than normal urinary tract tissue, but this result is not significant, possibly due to small sample size.

3) Lung primary squamous cell carcinoma xenograft models (the only squamous xenograft models with more than one sample available) show highly (and significantly) elevated SOST expression with respect to all other primary xenograft models in the tested collection. This confirms the finding in human primary (non-xenograft) tumors and is consistent with a recent literature report that describes a Wnt signature (including SOST upregulation) in squamous cell lung tumors (Hu et al., 2011, PLoS One, 6(10), e25807).

### Example 4

### Evaluation of SOST Expression in a Panel of Human Tumor Cell Lines and Primary Tumor Xenograft Samples

The expression of SOST was evaluated at both the mRNA and protein levels in a panel of cell lines and human primary xenograft samples.

For mRNA analysis, total RNA was isolated using an RNeasy Mini Kit (Qiagen, catalog #74106) and converted to cDNA using High Capacity cDNA Reverse Transcription Kit (Applied Biosystems, Inc., catalog #4368813). 3 μl cDNA was used per 10 μl QPCR reaction, which included 2X TaqMan Universal PCR Master Mix, Human B2M (beta-2-microglobulin) Endogenous Control and Human Sclerostin primer/probe sets (Applied Biosystems, catalog #4304437, 4310886E, Hs01072801_ml respectively). Real time PCR was carried out on an Applied Biosystems 7900HT Fast Real-Time PCR System with thermal profile: 95° C. for 10 min, and 40 cycles of 95° C. for 15 s/60° C. for 1 min. Threshold cycle numbers were used to determine mRNA expression relative to endogenous B2M control using the following formulae:

ΔC_{T}=C_{T}(B2M)−C_{T}(SOST) Relative Expression=2^{-ΔC}^{_{T}}

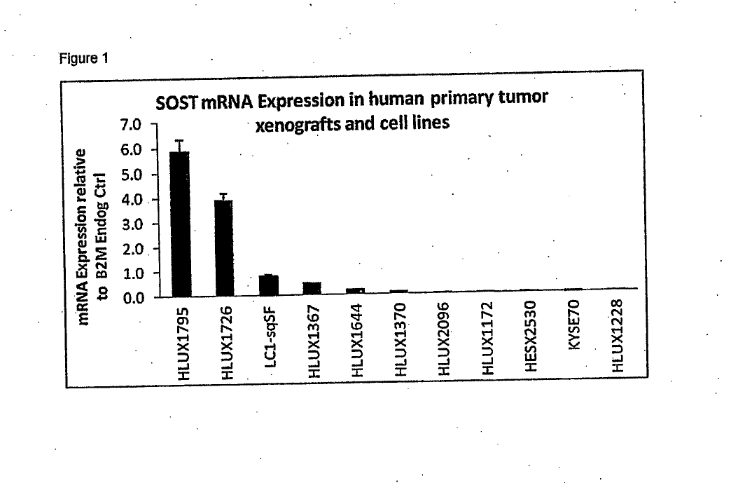

Results are presented in FIG. 1 and suggest that the human primary lung xenografts HLUX1795 and HLUX1726 have the highest (4-6 times higher) SOST mRNA expression compared to the other xenografts and cell lines measured.

Sclerostin (SOST) protein expression was evaluated by Western blotting. Frozen primary tumor xenografts or cell lines were treated with RIPA cell lysis/protein extraction buffer (50 mM Tris-HCl, 1% Triton X-100, 150 mM NaCl, 10% Glycerol, 0.25% Deoxycholate), supplemented with protease and phosphatase inhibitors (Roche catalog #11836153001, and 4906837001 respectively). Lysates were pelleted by centrifugation, cleared extract collected and quantified by BCA ELISA (Pierce Biotechnology, catalog #23227). 30 μg total protein was combined with reducing agent dithiothreitol, thermally denatured for 5 minutes at 85° C. and loaded into a 15-well, 4-12% Bis-Tris Gel, separated in MES Buffer for 55 minutes at 200 V, and transferred to 0.2 μm nitrocellulose membrane in 10% methanol for 1.5 hours at 40 V (Invitrogen, catalog #15508013, #NP0323BOX, NP0002, LC2000, respectively). The membrane was blocked for 1 h at room temperature with Odyssey Buffer (LiCor, catalog #927-40000), probed overnight at 4° C. with Human SOST Affinity Purified Polyclonal Ab, Goat IgG (R&D Systems, catalog #AF 1406), or Rabbit anti-Actin (Bethyl Laboratories, catalog #A300-485A), before incubation with HRP-conjugated secondary antibody (Jackson Immunoresearch, catalog #705-036-147, 111-036-045) for 1 h at room temperature. The membrane was then incubated in SuperSignal West Pico chemiluminescent substrate (Pierce, catalog #34078) and exposed to film (Actin, 10 second exposure; SOST, 5 min)

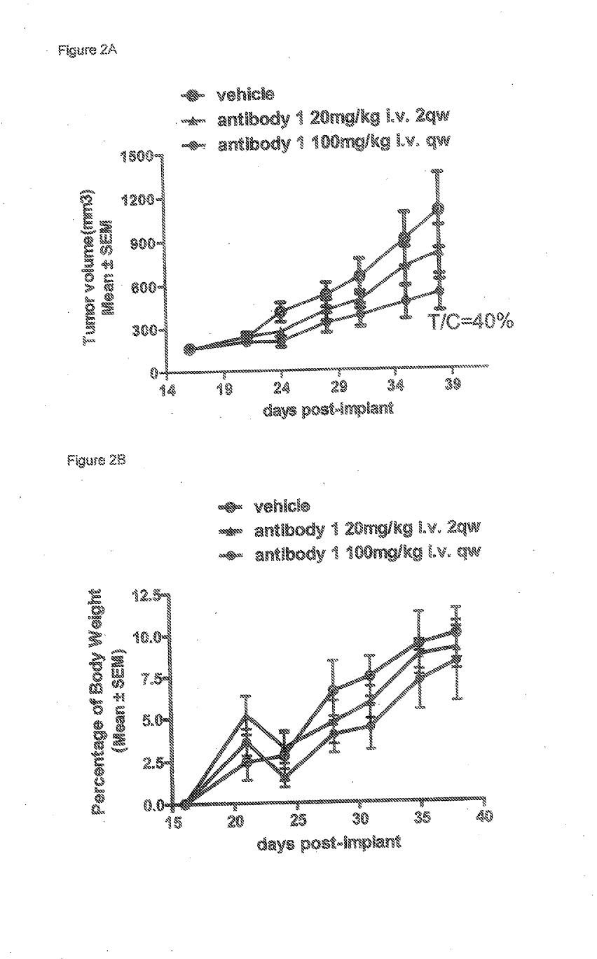

Results are presented in FIG. 2 and show that protein expression was observed in the cell line LC1sqSF and in human lung primary tumor xenografts HLUX1726, HLUX1644, HLUX1367 and the human esophageal primary tumor xenogrfat, HESX2530.

### Example 5

### Determination of in vivo Activity of Anti-SOST Antibodies

Anti-tumor activity of the anti-SOST antibody, Antibody 1, was evaluated in the HLUX1726 primary human tumor xenograft model. HLUX1726 tumor slurry in 100 μl 100% matrigel was implanted subcutaneously (s.c.) into female nude mice. Once tumors had reached 100-200 mm³, mice carrying HLUX1726 tumors (n=6 per group) were treated with vehicle IgG, or Antibody 1 (100 mg/kg, i.v., qw or 20 mg/kg, i.v. 2qw). Tumor growth was recorded twice a week using a digital caliper. Tumors treated with Antibody 1 displayed reduced growth relative to control tumors (T/C: 40%; FIG. 3A) and the treatment was well tolerated, as evidenced by differences in body weight between the Antibody 1 treated and control groups (FIG. 3B).

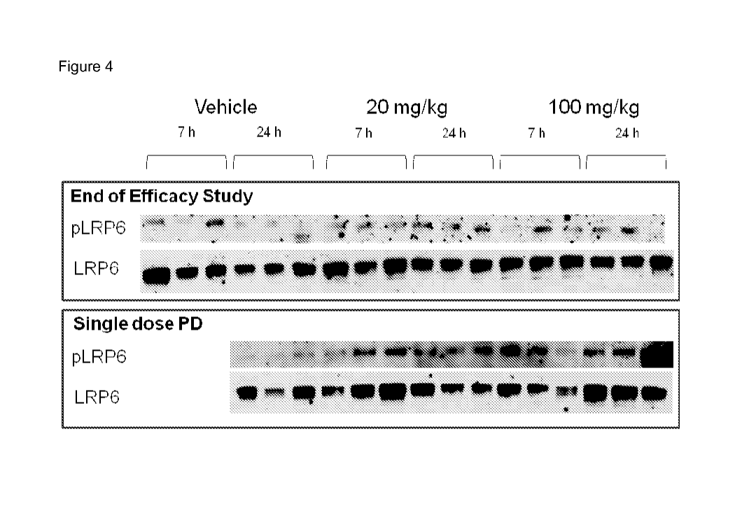

To measure the effect of the anti-SOST antibody, antibody 1, on Wnt signaling in the HLUX1726 tumors, mice implanted with HULX1726 tumors were dosed i.v. with a single dose of 20 or 100 mg/kg of the antibody and samples were taken at 7 and 24 h for phosphorylated LRP6 (pLRP6) evaluation by Western blot. Samples were also taken following the completion of efficacy studies, 7 and 24 h after the last doses of antibody 1. For Western blotting, total cell and primary human tumor xenograft lysates were prepared in RIPA buffer (50 mM Tris-HCl, pH 7.4, 150 mM NaCl, 1% NP-40, 0.5% sodium deoxycholate, 0.1% SDS, 1 mM EDTA). Lysates were normalized for protein concentration, resolved by SDS-PAGE, transferred onto nitrocellulose membranes and probed with an anti-pLRP6 antibody (rabbit anti-pS1490, Cell Signaling Technology, catalog #2568) with total LRP6 (Cell Signaling Technology, catalog #3395) used as a loading control. Results are presented in FIG. 4 and show more consistent phosphorylation of LRP6 in Antibody 1 treated samples either when collected 7 or 24 h after the final dose at the end of an anti-tumor efficacy study (upper panel) or after a single dose of the antibody (lower panel).

In addition, the anti-tumor effect of antibody 1 was evaluated under similar conditions to those described above in the HLUX1644 and LC1sqsf models. However, no significant anti-tumor activity was oberved in these models. The level of SOST protein expression was less in the HLUX1644 model relative to the HLUX1726 model (FIG. 2), which may explain the lack of effect of Antibody 1 in this model. In addition, it is worth noting that although LC1sqsf cells expressed similar levels of SOST protein as compared to the HLUX1726 model (FIG. 2), this is a cell line, rather than primary tumor model and the lack of effect may reflect a change in pathway dependency following growth on plastic as has been reported previously for developmental pathways (e.g. see Yauch et al., 2008, Nature, 455, 406)

In further studies, Antibodies 1, 2, 3 and 4 were evaluated in the HLUX1726 model. Briefly, HLUX1726 tumor slurry in 100 pi 100% matrigel was implanted subcutaneously (s.c.) into female nude mice. Once tumors had reached 100-200 mm³, mice carrying HLUX1726 tumors (n=6 per group) were treated with vehicle IgG, or Antibody 1, 2, 3 or 4. However, owing to aggregation of Antibodies 2, 3 and 4, all antibodies had to be dosed 100 mg/kg, i.p., qw rather than i.v. as in the previous study. Tumor growth was recorded twice a week using a digital caliper. Under these conditions, treatment with Antibody 1 resulted in a T/C of 64% and a T/C>90% was observed with Antibodies 2, 3 or 4.

1-56. (canceled)

57. A method of treating a squamous cell carcinoma (SCC), comprising administering an antagonist of scierostin to a patient in need thereof.

58. The method according to claim 57, wherein the SCC occurs in the lung, skin, lip, upper aerodigestive tract, urinary tract, esophagus, bladder, prostate, penis, vagina or cervix of the patient.

59. The method according to claim 58, wherein the SCC occurs in the upper aerodigestive tract, esophagus, urinary tract or lung.

60. The method according to claim 57, wherein the SCC occurs in the head or neck, larynx, mouth, pharynx, sinonasal cavity, nasal cavity, orbit, glottis, subglottis, supraglottis, alveolus, alveolus ridge, buccal mucosa, cheek, oral commissure, gingiva, lip, mandible, maxilla, maxillary antrum, mouth floor, mouth roof, paraoral area, retromolar space, oral sulcus, tongue, tonsillar fossa, uvula, oral vestibule, epiglottis, hypopharynx, nasopharnyx, orpharynx or sinus.

61. The method according to claim 57, wherein the antagonist of scierostin is an an scierostin antibody or antigen-binding fragment thereof.

62. The method according to claim 61, wherein the anti-scierostin antibody or antigen-binding fragment thereof is selected from the group consisting of
a. an anti-sclerostin antibody or antigen-binding fragment thereof comprising a heavy chain variable region comprising the amino acid sequence set forth as SEQ ID NO14;
b. an anti-scierostin antibody or antigen-binding fragment thereof comprising a light chain variable region comprising the amino acid sequence set forth as SEQ ID NO:5;
c. an anti-sclerostin antibody or antigen binding fragment thereof comprising a heavy chain variable region comprising the amino acid sequence set forth as SEQ ID NO:4 and a light chain variable region comprising the amino acid sequence set forth as SEQ ID NO:5; or
d. an anti-sclerostin antibody or antigen-binding fragment thereof comprising the three Complementarity-Determining Regions (CDRs) of the amino acid sequence set forth as SEQ ID NO:4 and the three CDRs of the amino add sequence set forth as SEQ ID NO:5.

63. The method according to claim 62, wherein the three CDRs of the amino acid sequence set forth as SEQ ID NO;4 are set forth in SEQ ID NO:6, SEQ. ID NO:7, and SEQ ID NO:8 and the three CDRs of the amino acid sequence set forth as SEQ ID NO:5 are set forth in SEQ ID NO:9, SEQ ID NO:10, and SEQ ID NO:11,

64. The method according to claim 61, wherein the anti-sclerostin antibody or antigen-binding fragment thereof is selected from the group consisting of:
a an anti-sclerostin antibody or antigen-binding fragment thereof comprising a heavy chain variable region comprising the amino acid sequence set forth as SEQ ID NO:14;
b. an anti-sclerostin antibody or antigen-binding fragment thereof comprising a light chain variable region comprising the amino acid sequence set forth as SEQ NO:15;
c. an anti-sclerostin antibody or antigen-binding fragment thereof comprising a heavy chain variable region comprising the amino acid sequence set forth as SEQ ID NO:14 and a light chain variable region comprising the amino acid sequence set forth as SEQ ID NO:15: or
d. an anti-sclerostin antibody or antigen-binding fragment thereof comprising the three CDRs of the amino acid sequence set forth as SEQ ID NO:14 and the three CDRs of the amino acid sequence set forth as SEQ ID NO:15.

65. The method according to claim 64, wherein the three CDRs of the amino acid sequence set forth as SEQ ID NO:14 are set forth in SEQ ID NO:16, SEQ ID NO:17, and SEQ ID NO:18 and the three CDRs of the amino acid sequence set forth as SEQ ID NO:15 are set forth in SEQ ID NO:19, SEQ ID NO:20, and SEQ ID NO:21.

66. The method according to claim 61, wherein the anti-sclerostin antibody or antigen-binding fragment thereof is selected from the group consisting of:
a. an anti-sclerostin antibody or antigen-binding fragment thereof comprising a heavy chain variable region comprising the amino acid sequence set forth as SEQ ID NO:24;
b. an anti-scierostin antibody or antigen-binding fragment thereof comprising a light chain variable region comprising the amino acid sequence set forth as SEQ ID NO:25;
c. an anti-sclerostin antibody or antigen-binding fragment thereof comprising a heavy chain variable region comprising the amino acid sequence set forth as SEQ ID NO:24 and a light chain variable region comprising the amino acid sequence set forth as SEQ ID NO:25; or
d. an anti-scierostin antibody or antigen-binding fragment thereof comprising the three CDRs of the amino acid sequence set forth as SEQ ID NO:24 and the three CDRs of the amino acid sequence set forth as SEQ ID NO:25.

67. The method according to claim 66, wherein the three CDRs of the amino acid sequence set forth as SEQ ID NO:24 are set forth in SEQ ID NO:26, SEQ ID NO:27, and SEQ ID NO:28 and the three CDRs of the amino acid sequence set forth as SEQ ID NO:25 are set forth in SEQ ID NO:29, SEQ ID NO:30, and SEQ ID NO:31.

68. The method according to claim 61, wherein the anti-sclerostin antibody or antigen-binding fragment thereof is selected from the group consisting at
a. an anti-solerostin antibody or antigen-binding fragment thereof comprising a heavy chain variable region comprising the amino acid sequence set forth as SEQ ID NO:34;
b. an anti-sclerostin antibody Or antigen-binding fragment thereof comprising a light chain variable region comprising the amino acid sequence set forth as SEQ ID NO:35;
c. an anti-sclerostin antibody or antigen-binding fragment thereof comprising a heavy chain variable region comprising the amino acid sequence set forth as SEQ ID NO:34 and a light chain variable region comprising the amino acid sequence set forth as SEQ ID NO:35; or
d. an anti-sclerostin antibody or antigen-binding fragment thereof comprising the three CDRs of the amino acid sequence set forth as SEQ ID NO:34 and the three CDRs of the amino acid sequence set forth as SEQ ID NQ:35.

69. The method according to claim 68, wherein the three CDRs of the amino acid sequence set forth as SEQ ID NO:34 are set forth in SEQ ID NO:36, SEQ ID NO:37, and SEQ ID NO:38 and the three CDRs of the amino acid sequence set forth as SEQ ID NO:35 are set forth in SEQ ID NO:39, SEQ ID NO:40, and SEQ ID NO:41.

70. The method according to claim 61, wherein the anti-sclerostin antibody or antigen-binding fragment thereof is selected from the group consisting of:
a an anti-sclerostin antibody or antigen-binding fragment thereof comprising a heavy chain variable region comprising the amino acid sequence set forth as SEQ ID NO:44;
b. an anti-sclerostin antibody or antigen-binding fragment thereof comprising a light chain variable region comprising the amino acid sequence set forth as SEQ ID NO:45;
c. an anti-sclerostin antibody or antigen-binding fragment thereof comprising a heavy chain variable region comprising the amino acid sequence set forth as SEQ ID NO:44 and a light chain variable region comprising the amino acid sequence set forth as SEQ ID NO:45; or
d. an anti-sclerostin antibody or antigen-binding fragment thereof comprising the three CDRs of the amino acid sequence set forth as SEQ ID NO:44 and the three CDRs of the amino acid sequence set forth as SEQ ID NO:45.

71. The method according to claim 70, wherein the three CDRs of the amino acid sequence set forth as SEQ ID NO:44 are set forth in SEQ ID NO:46, SEQ ID NO:47, and SEQ ID NO:48 and the three CDRs of the amino acid sequence set forth as SEQ ID NO:45 are set forth in SEQ ID NO:49, SEQ ID NO:50, and SEQ ID NO:51.
